(19) United States
(12) Patent Application Publication
(10) Pub. No.: US 2019/0112562 A1
(43) Pub. Date: Apr. 18, 2019

(54) ENZYMATIC CLEANING AND SANITIZING COMPOSITIONS AND METHODS OF USING THE SAME

(71) Applicant: Cinder Biological, Inc., Berkeley, CA (US)

(72) Inventors: Steven M. Yannone, Concord, CA (US); Jill O. Fuss, Oakland, CA (US); Adam Barnebey, Berkeley, CA (US)

(73) Assignee: Cinder Biological, Inc., Berkley, CA (US)

(21) Appl. No.: 16/091,074

(22) PCT Filed: Apr. 6, 2017
(86) PCT No.: PCT/US2017/026375

§ 371 Date: Oct. 3, 2018

Related U.S. Application Data
(60) Provisional application No. 62/319,213, filed on Apr. 6, 2016.

(51) Int. Cl. C11D 3/48; C11D 3/386; C11D 3/04; C11D 11/00; A61L 2/18; A01N 63/02; A01N 25/02

(52) U.S. Cl. CPC C11D 3/48; C11D 3/38618; C11D 3/38627; C11D 3/042; A61L 2202/17; C11D 11/0023; A61L 2/18; A01N 63/02; A01N 25/02; C11D 11/0041

(57) ABSTRACT

The present disclosure relates to a composition for cleaning or sanitizing a surface, methods of using the same for cleaning and sanitizing a surface, and kits comprising the same. The composition includes an enzyme isolated from a hyperthermophilic and/or acidophilic organism and optionally, an acid and an additive. The composition can be used at temperatures ranging from about 50° C. to 110° C., preferably at temperatures ranging from about 70° C. to 100° C. In addition, the composition can be used at pH values ranging from 0.5 to 7, preferably at pH values ranging from 2 to 5.

### STATEMENT OF GOVERNMENTAL SUPPORT

The subject matter described and claimed herein was support with funds supplied by the U.S. government in the form of a National Science Foundation (NSF) SBIR Phase I grant, awarded as Contract No. 1447398. The government may have certain rights to the subject matter disclosed herein.

### FIELD OF THE INVENTION

The present disclosure generally relates to compositions comprising a thermally stable and/or an acid stable enzyme isolated from a hyperthermophilic organism and an acid, and methods of using the same for cleaning and sanitation protocols.

### BACKGROUND

The food and beverage industries (e.g., production and serving) regularly clean and sanitize their industrial equipment to maintain product quality and ensure the public health. Production residuals that remain on industrial equipment compromise product quality and promote the growth of pathogenic microorganisms. Thus, the food and beverage industries typically clean and sanitize their industrial equipment to maintain functions and reduce microbial population to safe levels. Similarly, other industries that use fermentation processes to produce pharmaceuticals, cosmetics, nutritional supplements or biofuels also clean and sanitize their production equipment to maintain product quality and consistency.

However, cleaning and sanitation procedures can involve chemicals that are corrosive and toxic and that need to be properly disposed of in an environmentally sound manner as well as the use of large amounts of water for cleaning and rinsing equipment. Accordingly, there is a need for compositions and methods for cleaning and sanitizing equipment that are more compatible with the environment and reduce the amount of water consumed.

### SUMMARY

Provided herein is a composition for cleaning or sanitizing a vessel, wherein the composition includes an enzyme isolated from a hyperthermophilic organism and an acid. In some embodiments, the composition includes an acid selected from the group consisting of: nitric acid, phosphoric acid, hydrofluoric acid, sulfuric acid, hydrochloric acid, acetic acid, paracetic acid, citric acid, glycolic acid, formic acid, and mixtures or combinations thereof.

In some embodiments, the composition further includes a surfactant or detergent. In some embodiments, the surfactant or detergent is selected from the group consisting of: Brite Cleanse®, CHAPS, Pluronic® F-68, NP-40, sodium dodecyl sulfate (SDS), polysorbate 20, a saponin, Triton® X-100, sarkosyl, DetBuild®, and mixtures of combinations thereof.

In some embodiments, the enzyme included in the composition is isolated from an organism of the Archaea domain. In some embodiments, the enzyme is isolated from an organism of the Sulfolobales order.

In some embodiments, the enzyme included in the composition is selected from the group consisting of: a protease, a lipase, a cellulase, a hemicellulase, a glycoside hydrolase, an endoprotease, a carboxyesterase, an amylase, an alpha-amylase, an endoglucanase, an endopullulanase, a PNGase, a trehalase, a pullulanase, a peptidase, a signal peptidase, a xylanase, a cellobiohydrolase (CBH), a β-glucosidase, a peroxidase, a phospholipase, an esterase, a cutinase, a pectinase, a pectate lyase, a mannanase, a keratinase, a reductase, an oxidase, a phenoloxidase, a lipoxygenase, a ligninase, a tannase, a pentosanase, a malanase, a β-glucanase, an arabinosidase, a hyaluronidase, a chondroitinase, a laccase, a xyloglucanase, a xanthanase, an acyltransferase, a galactanase, a xanthan lyase, a xylanase, an arabinase, and combinations thereof.

In some embodiments, the composition further includes a food-safe additive as disclosed herein.

In some embodiments, the composition is effective for cleansing and/or sanitizing at a temperature of from about 50° C. to about 110° C. In some embodiments, the composition is effective for cleansing and/or sanitizing at a temperature of from about 60° C. to about 100° C. In some embodiments, the composition is effective for cleansing and/or sanitizing at a temperature of from about 70° C. to about 90° C., or from about 70° C. to about 85° C., or from about 75° C. to about 85° C., or from about 75° C. to about 80° C.

In some embodiments, the composition is effective for cleansing and/or sanitizing at a pH of from about 0.5 to about 7. In some embodiments, the composition is effective for cleansing and/or sanitizing at a pH of from about 0.5 to about 4.5, or from about 0.5 to about 3.0, or from about 0.5 to about 1.5. In some embodiments, the composition is effective for cleansing and/or sanitizing at a pH of from about 4 to about 7. In some embodiments, the composition is effective for cleansing and/or sanitizing at a pH of about 5.5. In some embodiments, the composition is effective for cleansing and/or sanitizing at a pH of about 3.0.

Embodiments are also directed to a method of cleaning a soiled surface, wherein the method includes: (a) providing a composition as disclosed herein; and (b) applying or contacting the composition with the surface, wherein the temperature of the composition upon application to the surface ranges from about 50° C. to about 110° C. and has a pH of from about 0.5 to about 7.0, and wherein the method results in at least about 25% of soil removal from the surface. In some embodiments, the composition is sprayed onto the surface. In some embodiments, the composition is poured onto the surface. In some embodiments, the surface is immersed in the composition.

In some embodiments directed to the method of cleaning, the method results in at least about 25% of soil removal from the surface. In some embodiments, the method results in at least about 30% of soil removal from the surface. In some embodiments, the method results in at least about 35% of soil removal from the surface. In some embodiments, the method results in at least about 40% of soil removal from the surface. In some embodiments, the method results in at least about 45%, 50%, 55%, 60%, 65%, 70%, 75%, 80%, 85%, 90%, or 95% of soil removal from the surface.

In some embodiments directed to the method of cleaning, the composition is applied to the surface for a duration of time ranging from about 5 minutes to about 120 minutes, or from about 10 minutes to about 100 minutes, or from about 20 minutes to about 90 minutes, or from about 30 minutes to about 75 minutes, or from about 40 minutes to about 60 minutes. In some embodiments, the composition is applied to the surface for a duration of time of at least about 45 minutes, or at least about 30 minutes, or at least about 20 minutes, or at least about 10 minutes. In some embodiments, the composition is applied to the surface for a duration of time of at least about 5 minutes.

In some embodiments, the method of cleaning further includes a step (c) of recovering the composition. In some embodiments, the method further includes a step (e) of applying or contacting the recovered composition with one or more additional surfaces to be cleaned. In some embodiments, steps (c) and (e) are repeated in succession at least once with one or more additional surfaces to be cleaned.

In some embodiments, the method of cleaning further includes a step (c) of recovering the composition. In some embodiments, the method further includes a step (d) of storing the composition for from about 30 minutes to about 15 days. In some embodiments, the method further includes a step (e) of applying or contacting the recovered composition with one or more additional surfaces to be cleaned. In some embodiments, steps (c), (d) and (e) are repeated in succession at least once with one or more additional surfaces to be cleaned.

In some embodiments, in step (d) of the method of cleaning, the composition is stored for from about 30 minutes to about 24 hours. In some embodiments, in step (d) of the method of cleaning, the composition is from about 1 day to about 15 days. In some embodiments, the composition is stored for about 1 day, about 3 days, about 5 days, about 7 days, about 10 days, or about 14 days.

In some embodiments, the method of cleaning is part of a clean-in-place (CIP) protocol. In some embodiments, the method of cleaning is part of a clean-out-of-place (COP) protocol.

Embodiments are also directed to a method of sanitizing a surface, wherein the method includes: (a) providing a composition as disclosed herein; and (b) applying or contacting the composition with the surface, wherein the temperature of the composition upon application to the surface ranges from about 50° C. to about 110° C. and has a pH of from about 0.5 to about 7.0, and wherein at least 95% of living organisms on the surface are eliminated and/or killed after applying the composition to the surface. In some embodiments, at least 97% of living organisms on the surface are eliminated and/or killed after applying the composition to the surface. In some embodiments, at least 99% of living organisms on the surface are eliminated and/or killed after applying the composition to the surface. In some embodiments, at least 99.5% of living organisms on the surface are eliminated and/or killed after applying the composition to the surface. The living organism can be at least one selected from the group consisting of: Enterococcus faecium, Streptococcus mutans, a Staphylococcus species, a Campylobacter species, a Clostridium species, a Bacillus species, an Enterobacter species, Listeria monocytogenes, E. coli O157:H7, Legionella pneumophila, Pseudomonas, Helicobacter pylori, Campylobacter jejuni, Clostridium perfringens, Clostridium difficile, Escherichia coli, Staphylococcus aureus, Salmonella spp., Salmonella typhimurium, Bacillus proteus, Bacillus subtilis, Bacillus cereus, Shigella spp., Streptococcus, Vibrio cholerae, Vibrio parahaemolyticus, Vibrio vulnificus, Yersinia enterocolitica, Yersinia pseudotuberculosis, Coxiella burnetii, Brucella spp., Corynebacterium ulcerans, Sarcinae spp. and Plesiomonas shigelloides.

In some embodiments of the method of sanitizing, the composition is sprayed onto the surface. In some embodiments, the composition is poured onto the surface. In some embodiments, the surface is immersed in the composition.

In some embodiments of the method of sanitizing, the composition is applied to the surface for a duration of time ranging from about 5 minutes to about 120 minutes, or from about 10 minutes to about 100 minutes, or from about 20 minutes to about 90 minutes, or from about 30 minutes to about 75 minutes, or from about 40 minutes to about 60 minutes. In some embodiments, the composition is applied to the surface for a duration of time of at least about 45 minutes, or at least about 30 minutes, or at least about 20 minutes, or at least about 10 minutes. In some embodiments, the composition is applied to the surface for a duration of time of at least about 5 minutes.

In some embodiments, the method of sanitizing further includes a step (c) of recovering the composition. In some embodiments, the method further includes a step (e) of applying or contacting the recovered composition with one or more additional surfaces to be sanitized and/or disinfected. In some embodiments, steps (c) and (e) are repeated in succession at least once with one or more additional surfaces to be sanitized and/or disinfected.

In some embodiments, the method of sanitizing further includes a step (c) of recovering the composition. In some embodiments, the method further includes a step (d) of storing the composition for from about 30 minutes to about 15 days. In some embodiments, the method further includes a step (e) of applying or contacting the recovered composition with one or more additional surfaces to be sanitized and/or disinfected. In some embodiments, steps (c), (d) and (e) are repeated in succession at least once with one or more additional surfaces to be sanitized and/or disinfected.

In some embodiments, in step (d) of the method of sanitizing, the composition is stored for from about 30 minutes to about 24 hours. In some embodiments, in step (d) of the method of sanitizing, the composition is stored for from about 1 day to about 15 days. In some embodiments, the composition is stored for about 1 day, about 3 days, about 5 days, about 7 days, about 10 days, or about 14 days.

In some embodiments, the method of sanitizing is part of a sanitize-in-place (SIP) protocol. In some embodiments, the method of sanitizing is part of a sanitize-out-of-place (SOP) protocol.

Embodiments are also directed to a kit for for cleaning or sanitizing a surface, wherein the kit includes: an enzyme or enzyme mixture, an acid, optionally one or more additives, and instructions for their use, wherein the enzyme or enzyme mixture is a hyperthermophilic and/or acidophilic enzyme or enzyme mixture as disclosed herein. In some embodiments, the enzyme or enzyme mixture is provided as a lyophilized product. In some embodiments, the enzyme or enzyme mixture is provided as a suspension. In some embodiments, the enzyme or enzyme mixture is provided as a solution.

In some embodiments directed to the kit, the enzyme or enzyme mixture, the acid and the optional additive(s) are provided in separate, individual containers. In some embodiments, the enzyme (or enzyme mixture) and the acid are provided in the same container, and the optional additive(s) are provided in a separate container. In some embodiments, the acid and optional additive(s) are provided in the same container, and the enzyme (or enzyme mixture) is provided in a separate container.

In some embodiments directed to the kit, the enzyme or enzyme mixture is provided in one container, and an optionally provided diluent is provided in a second, separate container. In some embodiments, instructions for preparing the enzyme or enzyme mixture in the optionally provided diluent are provided.

These and other embodiments of the invention along with many of its features are described in more detail in conjunction with the text below and attached figures.

### BRIEF DESCRIPTION OF THE FIGURES

FIG. 1 is a protein gel from a screen in which candidate protease enzymes were tested for activity in breaking down milk proteins.

FIG. 2 includes graphs that illustrate enzyme activity of exemplary protease enzymes as a function of operational pH (left) and temperature (right).

FIG. 3 is a bar graph that illustrates enzyme activity of an exemplary protease as a function of additives used in a dairy cleaning process.

FIG. 4 is a graph that illustrates enzyme activity of an exemplary protease as a function of concentration of various surfactants or detergents.

FIG. 5 is a bar chart of flux recovery measurements from fouled PES laboratory membranes after treatment with candidate enzymes.

FIG. 6 is a table that illustrates the improvements between the developed second generation cleaning protocol using enzyme formulations as disclosed herein and the manufacturers' recommended cleaning protocol.

FIG. 7 is an SDS-PAGE gel that was stained to indicate the presence of milk proteins (intact and degraded) at various time points during a dairy CIP process using candidate enzyme formulations.

FIG. 8 is a bar chart that indicates total protein in samples taken at various time points during a dairy CIP process using candidate enzyme formulations.

FIG. 9 is a photo of 316-grade stainless steel coupons that were employed for testing candidate enzymes in biofilm removal and sanitation protocols.

FIG. 10 is a photo of 316-grade stainless steel coupons with biofilm growth of various individual bacterial species.

FIG. 11 is a bar chart that illustrates coupon total bacterial cell count after treatment of 316-grade stainless steel coupons with and without candidate enzymes.

FIG. 12 includes graphs that illustrate percentages of enzyme activity retained under various storage conditions for two candidate enzymes.

FIG. 13 is a bar chart that illustrates enzyme activity of two candidate enzymes under standard storage and under lyophilized storage conditions at ambient temperatures.

FIG. 14 includes photos of SDS-PAGE gels (left) and of plates (right) that illustrate degradation of fats by two candidate lipase enzymes in the presence of detergent on a defined fatty acid (tributyrin) and on complex milk fats (ghee).

FIG. 15 is a bar chart that illustrates the inactivation of a dairy formulation by candidate protease enzymes at non-optimal temperatures and pH conditions.

### DETAILED DESCRIPTION OF THE EMBODIMENTS

Previously, a suite of enzymes that function optimally extreme temperatures and highly acidic conditions was described (WO 2014/081973). Disclosed herein are compositions comprising acid- and heat-stable enzymes and methods of using the same for sanitation and cleaning applications under extreme heat and acidic conditions in combination with detergents, surfactants and/or other chemical additives. The efficacy of combined thermal/acid/enzyme treatments in for membrane defouling and biofilm sanitation and biofilm residue removal that can inhibit recolonization is also demonstrated. Applications for biofilm sanitation in medical and dental equipment, food processing facilities and equipment, using ultra-stable enzymes in combination with heat and/ or acid and/or detergents and surfactants as well as other chemical additives are also identified.

### Definitions

Unless defined otherwise, all technical and scientific terms used herein have the same meaning as is commonly understood by one of ordinary skill in the art. In the event that there is a plurality of definitions for a term herein, those in this section prevail unless stated otherwise.

As used herein, the singular forms “a,” “an,” and “the” include the plural reference unless the context clearly indicates otherwise. Thus, for example, reference to an “an enzyme” is a reference to one or more enzymes, etc.

As used herein, the term “isolated” refers to an enzyme that is substantially or essentially free of components that normally accompany or interact with the enzyme as found in its naturally occurring environment or in its production environment, or both. Isolated enzyme preparations have less than about 30%, less than about 25%, less than about 20%, less than about 15%, less than about 10%, less than about 5%, less than about 4%, less than about 3%, less than about 2% or less than about 1% of contaminating protein by weight, e.g. dry weight.

The term “stable” in reference to an enzyme relates to the enzyme's ability to retain its function and/or activity over time. The term “stable” is used herein as a relative term to compare the enzyme's ability to retain its function and/or activity over time in two or more different states or conditions. For example, a hyperthermophilic and/or acidophilic enzyme is referred to as being stable under high temperature and/or low pH conditions in comparison to a condition when the enzyme is not in those conditions. In some embodiments, an enzyme is stable if it retains at least about 50%, 55%, 60%, 65%, 75%, 80%, 85%, 90%, 95%, 98%, 99%, or any amount included between any two of these values, of its function and/or activity over time.

The term “half-life” of an enzyme typically refers to the time required for the activity of an enzyme to be reduced by one-half.

The term “thermophilic” refers to an organism, entity or component which is capable of growth and/or survival, or exhibits activity, at temperatures ranging from about 50° C. to about 110° C. Accordingly, a thermophilic organism is capable of growth and/or survival at temperatures of about 50° C., 55° C., 60° C., 65° C., 70° C., 75° C., 80° C., 85° C., 90° C., 95° C., 100° C., 105° C., 110° C., or at any temperature included between any two of these values. Similarly, a thermophilic enzyme exhibits activity at temperatures of about 50° C., 55° C., 60° C., 65° C., 70° C., 75° C., 80° C., 85° C., 90° C., 95° C., 100° C., 105° C., 110° C., or at any temperature included between any two of these values. In some embodiments, a thermophilic enzyme exhibits at least about 10% of its maximum activity at temperatures of about 50° C., 55° C., 60° C., 65° C., 70° C., 75° C., 80° C., 85° C., 90° C., 95° C., 100° C., 105° C., 110° C., or at any temperature included between any two of these values. In some embodiments, a thermophilic enzyme exhibits at least about 15% of its maximum activity at temperatures of about 50° C., 55° C., 60° C., 65° C., 70° C., 75° C., 80° C., 85° C., 90° C., 95° C., 100° C., 105° C., 110° C., or at any temperature included between any two of these values. In some embodiments, a thermophilic enzyme exhibits at least about 20% of its maximum activity at temperatures of about 50° C., 55° C., 60° C., 65° C., 70° C., 75° C., 80° C., 85° C., 90° C., 95° C., 100° C., 105° C., 110° C., or at any temperature included between any two of these values. In some embodiments, a thermophilic enzyme exhibits at least about 25% of its maximum activity at temperatures of about 50° C., 55° C., 60° C., 65° C., 70° C., 75° C., 80° C., 85° C., 90° C., 95° C., 100° C., 105° C., 110° C., or at any temperature included between any two of these values. In some embodiments, a thermophilic enzyme exhibits at least about 30%, 35%, 40%, 45%, 50%, 55%, 60%, 65%, or 75% of its maximum activity, or any percent activity included between any two of these values, at temperatures of about 50° C., 55° C., 60° C., 65° C., 70° C., 75° C., 80° C., 85° C., 90° C., 95° C., 100° C., 105° C., 110° C., or at any temperature included between any two of these values. In some embodiments, “thermophilic” refers to an entity or component which is capable of growth and/or survival, or exhibits activity, at temperatures ranging from about 65° C. to about 100° C., or from about 70° C. to about 95° C., or from about 75° C. to about 90° C., or any range included between and including any two of these values. This is in contrast to mesophilic organisms or components, which in general are capable of growth and/or survival, or exhibits activity, at temperatures ranging from about 30° C. to 37° C.

The term “hyperthermophilic” refers to an organism, entity or component which is capable of growth and/or survival, or exhibits activity, at temperatures ranging from about 70° C. to about 110° C. Accordingly, a hyperthermophilic organism is capable of growth at temperatures of about 70° C., 75° C., 80° C., 85° C., 90° C., 95° C., 100° C., 105° C., 110° C., or at any temperature included between any two of these values. Similarly, a hyperthermophilic enzyme exhibits activity at temperatures of about 70° C., 75° C., 80° C., 85° C., 90° C., 95° C., 100° C., 105° C., 110° C., or at any temperature included between any two of these values. In some embodiments, a thermophilic enzyme exhibits at least about 10% of its maximum activity at temperatures of about 70° C., 75° C., 80° C., 85° C., 90° C., 95° C., 100° C., 105° C., 110° C., or at any temperature included between any two of these values. In some embodiments, a thermophilic enzyme exhibits at least about 15% of its maximum activity at temperatures of about 70° C., 75° C., 80° C., 85° C., 90° C., 95° C., 100° C., 105° C., 110° C., or at any temperature included between any two of these values. In some embodiments, a thermophilic enzyme exhibits at least about 20% of its maximum activity at temperatures of about 70° C., 75° C., 80° C., 85° C., 90° C., 95° C., 100° C., 105° C., 110° C., or at any temperature included between any two of these values. In some embodiments, a thermophilic enzyme exhibits at least about 25% of its maximum activity at temperatures of about 70° C., 75° C., 80° C., 85° C., 90° C., 95° C., 100° C., 105° C., 110° C., or at any temperature included between any two of these values. In some embodiments, a thermophilic enzyme exhibits at least about 30%, 35%, 40%, 45%, 50%, 55%, 60%, 65%, or 75% of its maximum activity, or any percent activity included between any two of these values, at temperatures of about 70° C., 75° C., 80° C., 85° C., 90° C., 95° C., 100° C., 105° C., 110° C., or at any temperature included between any two of these values. In some embodiments, “hyperthermophilic” refers to an entity or component which is capable of growth and/or survival, or exhibits activity, at temperatures ranging from about 70° C. to about 105° C., or from about 75° C. to about 100° C., or from about 80° C. to about 95° C., or any range included between and including any two of these values. The term “thermophilic” can, in this context, cover “hyperthermophilic” organisms and components as well.

As used herein, the term “acidophilic” refers to an organism, entity or component which is capable of growth and/or survival, or exhibits activity, at pH values ranging from about 0.5 to about 5.5. Accordingly, an acidophilic organism is capable of growth at pH values of about 0.5, 1.0, 1.5, 2.0, 2.5, 3.0, 3.5, 4.0, 4.5, 5.0, 5.5, or at any pH value included between any two of these values. Similarly, an acidophilic enzyme exhibits activity at pH values of about 0.5, 1.0, 1.5, 2.0, 2.5, 3.0, 3.5, 4.0, 4.5, 5.0, 5.5, or at any pH value included between any two of these values. For example, in some embodiments, an acidophilic enzyme exhibits at least about 10% of its maximum activity at pH values of about 0.5, 1.0, 1.5, 2.0, 2.5, 3.0, 3.5, 4.0, 4.5, 5.0, 5.5, or at any pH value included between any two of these values. In some embodiments, an acidophilic enzyme exhibits at least about 15% of its maximum activity at pH values of about 0.5, 1.0, 1.5, 2.0, 2.5, 3.0, 3.5, 4.0, 4.5, 5.0, 5.5, or at any pH value included between any two of these values. In some embodiments, an acidophilic enzyme exhibits at least about 20% of its maximum activity at pH values of about 0.5, 1.0, 1.5, 2.0, 2.5, 3.0, 3.5, 4.0, 4.5, 5.0, 5.5, or at any pH value included between any two of these values. In some embodiments, an acidophilic enzyme exhibits at least about 25% of its maximum activity at pH values of about 0.5, 1.0, 1.5, 2.0, 2.5, 3.0, 3.5, 4.0, 4.5, 5.0, 5.5, or at any pH value included between any two of these values. In some embodiments, an acidophilic enzyme exhibits at least about 30%, 35%, 40%, 45%, 50%, 55%, 60%, 65%, or 75% of its maximum activity, or any percent activity included between any two of these values, at pH values of about 0.5, 1.0, 1.5, 2.0, 2.5, 3.0, 3.5, 4.0, 4.5, 5.0, 5.5, or at any pH value included between any two of these values. In some embodiments, an “acidophilic” entity or component is capable of growth and/or survival, or exhibits activity, at pH values ranging from about 0.5 to about 3.5, or from about 0.5 to about 2.5, or from about 0.5 to about 1.5, or from about 2.0 to about 3.0, or any range included between and including any two of these values. In some embodiments, an “acidophilic” entity or component is capable of growth and/or survival, or exhibits activity, at pH values ranging from about 2.0 to about 5.0, or from about 3.0 to about 5.0, or from about 4.0 to about 5.0, or or any range included between and including any two of these values.

As used herein, the terms “cleaning” or “cleansing” refer to a procedure that reduces the amount of soil on a soiled surface. In some embodiments, “cleaning” or “cleansing” reduces the amount of soil on the soiled surface by about 10%, about 20%, about 30%, about 40%, about 50%, about 60%, about 70%, about 80%, about 90%, about 95%, about 99%, about 100%, or by any amount included between any two of these values. The extent to which a surface is cleaned or cleansed can be measured by any procedure known to one of ordinary skill in the art.

Similarly, the terms “cleaner,” “cleaning composition,” or “cleaning agent” refer to a composition or agent that, when added to a soiled surface (e.g., industrial equipment), reduces the amount of soil on a soiled surface. In some embodiments, the “cleaner,” “cleaning composition,” or “cleaning agent” reduces the amount of soil on the soiled substrate (i.e., cleans the substrate) by about 10%, about 20%, about 30%, about 40%, about 50%, about 60%, about 70%, about 80%, about 90%, about 95%, about 99%, about 100%, or by any amount included between any two of these values. The extent to which the “cleaner,” “cleaning composition,” or “cleaning agent” reduces the amount of soil on the soiled substrate can be measured by any procedure known to one of ordinary skill in the art.

As used herein, the term “sanitizing” refers to the reduction of microbial populations to safe levels established by public health regulations. A sanitized surface is, as defined by the Environmental Protection Agency (EPA), a consequence of a process or program containing both an initial cleaning and a subsequent sanitizing treatment which must be separated by a potable water rinse. A sanitizing treatment applied to a cleaned food contact surface must result in a reduction in population of at least 99.999% (5 log) for specified microorganisms as defined by the “Germicidal and Detergent Sanitizing Action of Disinfectants”, Official Methods of Analysis of the Association of Official Analytical Chemists, paragraph 960.09 and applicable sections, 15th Edition, 1990 (EPA Guideline 91-2).

As used herein, the term “soil” refers to any foreign substance that is in contact with a surface. In some embodiments, the soil on a soiled surface includes, but is not limited to, a residue of a grain, a dairy product, an alcoholic beverage, a non-alcoholic beverage, a fruit, a vegetable, a meat, an animal food, a soiled dish residue, an industrial fermentation product, an algae, a biofuel, a pharmaceutical, a nutritional supplement, a cosmetic or a combination of any two or more thereof.

As used herein, the term “hyperthermophilic acidophile” refers to an organism or entity which is capable of growth and/or survival (1) at temperatures ranging from about 70° C. to about 110° C., and (2) at pH values ranging from about 4.0 to about 6.5. In some embodiments, a hyperthermophilic acidophile is capable of growth and/or survival (1) at temperatures of about 70° C., 75° C., 80° C., 85° C., 90° C., 95° C., 100° C., 105° C., 110° C., or at any temperature included between any two of these values, and (2) at pH values of about 4.0, 4.25, 4.5, 4.75, 5.0, 5.25, 5.5, 5.75, 6.0, 6.25, 6.5, or at any pH value included between any two of these values. In some embodiments, a hyperthermophilic acidophile is capable of growth and/or survival (1) at temperatures of about 70° C., 75° C., 80° C., 85° C., 90° C., 95° C., or at any temperature included between any two of these values, and (2) at pH values of about 5.0, 5.25, 5.5, 5.75, 6.0 or at any pH value included between any two of these values. Enzymes isolated or obtained from hyperthermophilic acidophiles can exhibit activity at any of the foregoing temperature and pH ranges suitable for hyperthemophilic acidophile growth and/or survival.

Compositions

Embodiments relate to a composition useful for cleaning and/or sanitizing a surface. Generally, the compositions comprise a thermally stable and/or an acid stable enzyme isolated from a hyperthermophilic organism. In some embodiments, the compositions also contain an acid.

In some embodiments, the composition has a pH value ranging from about 0.5 to about 7. In some embodiments, the compositions have a pH value ranging from about 0.5 to about 4.5, or from about 0.5 to about 3.0, or from about 0.5 to about 1.5, or any pH range included between and including any two of these values. In some embodiments, the composition has a pH of about 2.0 to 3.0. In some embodiments, the composition has a pH value ranging from about 4 to about 7, or from about 4.5 to about 6.5, or from about 5 to about 6, or any pH range included between and including any two of these values. In some embodiments, the composition has a pH of about 5.5. In some embodiments, the composition has a pH of about 3.0.

Also provided herein are compositions as disclosed herein that can be applied to a surface under pH conditions ranging from about 0.5 to about 7. In some embodiments, the composition can be applied to a surface under pH conditions ranging from about 0.5 to about 4.5, or from about 0.5 to about 3.0, or from about 0.5 to about 1.5, or any pH range included between and including any two of these values. In some embodiments, the composition can be applied to a surface under pH conditions ranging from about 4 to about 7, or from about 4.5 to about 6.5, or from about 5 to about 6, or any pH range included between and including any two of these values. In some embodiments, the composition can be applied to a surface at a pH condition of about 5.5. In some embodiments, the composition can be applied to a surface at a pH condition of about 3.0.

In some embodiments, the compositions disclosed herein can be employed at temperatures ranging from about 50° C. to about 110° C. For example the compositions can be applied to a surface at temperature conditions of about 50° C., 55° C., 60° C., 65° C., 70° C., 75° C., 80° C., 85° C., 90° C., 95° C., 100° C., 105° C., 110° C., or any temperature included between any two of these values.

In some embodiments, the compositions disclosed herein are heated to temperatures ranging from about 50° C. to about 110° C. prior to application to a surface. For example the compositions can be heated to a temperature of about 50° C., 55° C., 60° C., 65° C., 70° C., 75° C., 80° C., 85° C., 90° C., 95° C., 100° C., 105° C., 110° C., or any temperature included between any two of these values. Once the composition reaches its target temperature within this range, it can be employed as part of a method to clean or sanitize a surface.

Enzymes

Any enzyme or mixture of enzymes, from a source that is hyperthermophilic and/or acidophilic, can be provided in the composition, provided that the enzyme or mixture of enzymes is stable in the desired pH range and compatible with the compositions and operating conditions disclosed herein. In some embodiments, the enzyme can be an enzyme isolated and/or produced in a manner described in WO 2014/081973, which is incorporated herein by reference in its entirety. In some embodiments, the enzyme is provided in a solid form, a liquid form, or a lyophilized form.

The enzyme can be provided in an amount that is effective for cleaning or sanitizing a surface. In some embodiments, the enzyme is provided in an amount of from about 0.0001 mg to 1000 mg of enzyme protein, or from about 0.001 mg to 750 mg of enzyme protein, or from about 0.01 mg to 500 mg of enzyme protein, or from about 0.05 mg to 250 mg of enzyme protein, or from about 0.2 mg to 100 mg of enzyme protein, or from about 0.5 mg to 50 mg of enzyme protein per 100 grams of soil on the surface, or any amount included between any two of these values. For example, the amount of enzyme can be about 0.1 mg, 0.25 mg, 0.5 mg, 1 mg, 2.5 mg, 5 mg. 10 mg, 25 mg, 50 mg, 100 mg, 250 mg, 500 mg, 1000 mg, or any amount included between any two of these values, of enzyme protein per 100 grams of soil on the surface.

In some embodiments, the enzyme is provided in a concentration that ranges from about 0.0001 wt % to 50 wt %, or from about 0.001 wt % to 40 wt %, or from about 0.01 wt % to 30 wt %, or from about 0.1 wt % to 25 wt %, or from about 0.5 wt % to 20 wt %, or from about 1 wt % to 15 wt %, or from about 2.5 wt % to 10 wt %, or any range included between and including any two of these values. In some embodiments, the enzyme is provided in a concentration of about 0.0001 wt %, 0.001 wt %, 0.01 wt %, 0.1 wt %, 0.25 wt %, 0.5 wt %, 1 wt %, 2 wt %, 2.5 wt %, 3 wt %, 4 wt %, 5 wt %, 10 wt %, 15 wt %, 20 wt %, 25 wt %, 30 wt %, 35 wt %, 40 wt %, 45 wt %, 50 wt %, or any value included between any two of these values.

In some embodiments, the enzyme is provided in an activity range of from about 0.0001 to 100 activity units, or from about 0.001 to 75 activity units, or from about 0.01 to 50 activity units, or from about 0.1 to 25 activity units, or from about 0.5 to 20 activity units, or from about 1 to 15 activity units, or from about 2.5 to 10 activity units, or any range included between and including any two of these values. In some embodiments, the enzyme is provided in an amount of about 0.0001 activity unit, 0.001 activity unit, 0.01 activity unit, 0.1 activity unit, 0.25 activity unit, 0.5 activity unit, 1 activity unit, 2 activity units, 2.5 activity units, 3 activity units, 4 activity units, 5 activity units, 10 activity units, 15 activity units, 20 activity units, 25 activity units, 30 activity units, 35 activity units, 40 activity units, 45 activity units, 50 activity units, 75 activity units, 100 activity units, or amount included between any two of these values.

In some embodiments, the enzyme or enzyme mixture is stable in a pH range of from about 0.5 to about 7. In some embodiments, the enzyme or enzyme mixture is stable in a pH range of from about 0.5 to about 4.5, or from about 0.5 to about 3.0, or from about 0.5 to about 1.5, or any pH range included between and including any two of these values. In some embodiments, the enzyme or enzyme mixture is stable in a pH range of from about 2.0 to 3.0. In some embodiments, the enzyme or enzyme mixture is stable in a pH range of from about 4 to about 7, or from about 4.5 to about 6.5, or from about 5 to about 6, or any pH range included between and including any two of these values. In some embodiments, the enzyme or enzyme mixture is stable at a pH of about 5.5. In some embodiments, the enzyme or enzyme mixture is stable at a pH of about 3.0.

In some embodiments, the enzyme or enzyme mixture demonstrates enzymatic activity in a pH range of from about 0.5 to about 7. In some embodiments, the enzyme or enzyme mixture demonstrates enzymatic activity in a pH range of from about 0.5 to about 4.5, or from about 0.5 to about 3.0, or from about 0.5 to about 1.5, or any pH range included between and including any two of these values. In some embodiments, the enzyme or enzyme mixture demonstrates enzymatic activity in a pH range of from about 2.0 to 3.0. In some embodiments, the enzyme or enzyme mixture demonstrates enzymatic activity in a pH range of from about 4 to about 7, or from about 4.5 to about 6.5, or from about 5 to about 6, or any pH range included between and including any two of these values. In some embodiments, the enzyme or enzyme mixture demonstrates enzymatic activity at a pH of about 5.5. In some embodiments, the enzyme or enzyme mixture demonstrates enzymatic activity at a pH of about 3.0.

In some embodiments, the enzyme or enzyme mixture demonstrates optimal enzymatic activity in a pH range of from about 0.5 to about 7. In some embodiments, the enzyme or enzyme mixture demonstrates optimal enzymatic activity in a pH range of from about 0.5 to about 4.5, or from about 0.5 to about 3.0, or from about 0.5 to about 1.5, or any pH range included between and including any two of these values. In some embodiments, the enzyme or enzyme mixture demonstrates optimal enzymatic activity in a pH range of from about 2.0 to 3.0. In some embodiments, the enzyme or enzyme mixture demonstrates optimal enzymatic activity in a pH range of from about 4 to about 7, or from about 4.5 to about 6.5, or from about 5 to about 6, or any pH range included between and including any two of these values. In some embodiments, the enzyme or enzyme mixture demonstrates optimal enzymatic activity at a pH of about 5.5. In some embodiments, the enzyme or enzyme mixture demonstrates optimal enzymatic activity at a pH of about 3.0.

In some embodiments, the enzyme or enzyme mixture demonstrates at least about 10% of its maximum enzymatic activity in a pH range of from about 0.5 to about 7. In some embodiments, the enzyme or enzyme mixture demonstrates at least about 10% of its maximum enzymatic activity in a pH range of from about 0.5 to about 4.5, or from about 0.5 to about 3.0, or from about 0.5 to about 1.5, or any pH range included between and including any two of these values. In some embodiments, the enzyme or enzyme mixture demonstrates at least about 10% of its maximum enzymatic activity in a pH range of from about 2.0 to 3.0. In some embodiments, the enzyme or enzyme mixture demonstrates at least about 10% of its maximum enzymatic activity in a pH range of from about 4 to about 7, or from about 4.5 to about 6.5, or from about 5 to about 6, or any pH range included between and including any two of these values. In some embodiments, the enzyme or enzyme mixture demonstrates at least about 10% of its maximum enzymatic activity at a pH of about 5.5. In some embodiments, the enzyme or enzyme mixture demonstrates at least about 10% of its maximum enzymatic activity at a pH of about 3.0.

In some embodiments, the enzyme or enzyme mixture demonstrates at least about 15% of its maximum enzymatic activity in a pH range of from about 0.5 to about 7. In some embodiments, the enzyme or enzyme mixture demonstrates at least about 15% of its maximum enzymatic activity in a pH range of from about 0.5 to about 4.5, or from about 0.5 to about 3.0, or from about 0.5 to about 1.5, or any pH range included between and including any two of these values. In some embodiments, the enzyme or enzyme mixture demonstrates at least about 15% of its maximum enzymatic activity in a pH range of from about 2.0 to 3.0. In some embodiments, the enzyme or enzyme mixture demonstrates at least about 15% of its maximum enzymatic activity in a pH range of from about 4 to about 7, or from about 4.5 to about 6.5, or from about 5 to about 6, or any pH range included between and including any two of these values. In some embodiments, the enzyme or enzyme mixture demonstrates at least about 15% of its maximum enzymatic activity at a pH of about 5.5. In some embodiments, the enzyme or enzyme mixture demonstrates at least about 15% of its maximum enzymatic activity at a pH of about 3.0.

In some embodiments, the enzyme or enzyme mixture demonstrates at least about 20% of its maximum enzymatic activity in a pH range of from about 0.5 to about 7. In some embodiments, the enzyme or enzyme mixture demonstrates at least about 20% of its maximum enzymatic activity in a pH range of from about 0.5 to about 4.5, or from about 0.5 to about 3.0, or from about 0.5 to about 1.5, or any pH range included between and including any two of these values. In some embodiments, the enzyme or enzyme mixture demonstrates at least about 20% of its maximum enzymatic activity in a pH range of from about 2.0 to 3.0. In some embodiments, the enzyme or enzyme mixture demonstrates at least about 20% of its maximum enzymatic activity in a pH range of from about 4 to about 7, or from about 4.5 to about 6.5, or from about 5 to about 6, or any pH range included between and including any two of these values. In some embodiments, the enzyme or enzyme mixture demonstrates at least about 20% of its maximum enzymatic activity at a pH of about 5.5. In some embodiments, the enzyme or enzyme mixture demonstrates at least about 20% of its maximum enzymatic activity at a pH of about 3.0.

In some embodiments, the enzyme or enzyme mixture demonstrates at least about 25% of its maximum enzymatic activity in a pH range of from about 0.5 to about 7. In some embodiments, the enzyme or enzyme mixture demonstrates at least about 25% of its maximum enzymatic activity in a pH range of from about 0.5 to about 4.5, or from about 0.5 to about 3.0, or from about 0.5 to about 1.5, or any pH range included between and including any two of these values. In some embodiments, the enzyme or enzyme mixture demonstrates at least about 25% of its maximum enzymatic activity in a pH range of from about 2.0 to 3.0. In some embodiments, the enzyme or enzyme mixture demonstrates at least about 25% of its maximum enzymatic activity in a pH range of from about 4 to about 7, or from about 4.5 to about 6.5, or from about 5 to about 6, or any pH range included between and including any two of these values. In some embodiments, the enzyme or enzyme mixture demonstrates at least about 25% of its maximum enzymatic activity at a pH of about 5.5. In some embodiments, the enzyme or enzyme mixture demonstrates at least about 25% of its maximum enzymatic activity at a pH of about 3.0.

In some embodiments, the enzyme or enzyme mixture demonstrates at least about 30%, 35%, 40%, 45%, 50%, 55%, 60%, 65%, or 75% of its maximum activity, or any percent activity included between any two of these values, in a pH range of from about 0.5 to about 7. In some embodiments, the enzyme or enzyme mixture demonstrates at least about 30%, 35%, 40%, 45%, 50%, 55%, 60%, 65%, or 75% of its maximum activity, or any percent activity included between any two of these values, in a pH range of from about 0.5 to about 4.5, or from about 0.5 to about 3.0, or from about 0.5 to about 1.5, or any pH range included between and including any two of these values. In some embodiments, the enzyme or enzyme mixture demonstrates at least about 30%, 35%, 40%, 45%, 50%, 55%, 60%, 65%, or 75% of its maximum activity, or any percent activity included between any two of these values, in a pH range of from about 2.0 to 3.0. In some embodiments, the enzyme or enzyme mixture demonstrates at least about 30%, 35%, 40%, 45%, 50%, 55%, 60%, 65%, or 75% of its maximum activity, or any percent activity included between any two of these values, in a pH range of from about 4 to about 7, or from about 4.5 to about 6.5, or from about 5 to about 6, or any pH range included between and including any two of these values. In some embodiments, the enzyme or enzyme mixture demonstrates at least about 30%, 35%, 40%, 45%, 50%, 55%, 60%, 65%, or 75% of its maximum activity, or any percent activity included between any two of these values, at a pH of about 5.5. In some embodiments, the enzyme or enzyme mixture demonstrates at least about 30%, 35%, 40%, 45%, 50%, 55%, 60%, 65%, or 75% of its maximum activity, or any percent activity included between any two of these values, at a pH of about 3.0.

In some embodiments, the enzyme or enzyme mixture demonstrates enzymatic activity at temperatures ranging from about 50° C. to about 110° C. For example, the enzyme or enzyme mixture demonstrates enzymatic activity at temperature conditions of about 50° C., 55° C., 60° C., 65° C., 70° C., 75° C., 80° C., 85° C., 90° C., 95° C., 100° C., 105° C., 110° C., or any temperature included between any two of these values. In some embodiments, the enzyme or enzyme mixture demonstrates optimal enzymatic activity at temperature conditions of about 50° C., 55° C., 60° C., 65° C., 70° C., 75° C., 80° C., 85° C., 90° C., 95° C., 100° C., 105° C., 110° C., or any temperature included between any two of these values.

In some embodiments, the enzyme or enzyme mixture demonstrates at least about 10% of its maximum enzymatic activity at temperatures ranging from about 50° C. to about 110° C. For example, the enzyme or enzyme mixture demonstrates at least about 10% of its maximum enzymatic activity at temperature conditions of about 50° C., 55° C., 60° C., 65° C., 70° C., 75° C., 80° C., 85° C., 90° C., 95° C., 100° C., 105° C., 110° C., or any temperature included between any two of these values.

In some embodiments, the enzyme or enzyme mixture demonstrates at least about 15% of its maximum enzymatic activity at temperatures ranging from about 50° C. to about 110° C. For example, the enzyme or enzyme mixture demonstrates at least about 15% of its maximum enzymatic activity at temperature conditions of about 50° C., 55° C., 60° C., 65° C., 70° C., 75° C., 80° C., 85° C., 90° C., 95° C., 100° C., 105° C., 110° C., or any temperature included between any two of these values.

In some embodiments, the enzyme or enzyme mixture demonstrates at least about 20% of its maximum enzymatic activity at temperatures ranging from about 50° C. to about 110° C. For example, the enzyme or enzyme mixture demonstrates at least about 20% of its maximum enzymatic activity at temperature conditions of about 50° C., 55° C., 60° C., 65° C., 70° C., 75° C., 80° C., 85° C., 90° C., 95° C., 100° C., 105° C., 110° C., or any temperature included between any two of these values.

In some embodiments, the enzyme or enzyme mixture demonstrates at least about 25% of its maximum enzymatic activity at temperatures ranging from about 50° C. to about 110° C. For example, the enzyme or enzyme mixture demonstrates at least about 25% of its maximum enzymatic activity at temperature conditions of about 50° C., 55° C., 60° C., 65° C., 70° C., 75° C., 80° C., 85° C., 90° C., 95° C., 100° C., 105° C., 110° C., or any temperature included between any two of these values.

In some embodiments, the enzyme or enzyme mixture demonstrates at least about 30%, 35%, 40%, 45%, 50%, 55%, 60%, 65%, or 75% of its maximum activity, or any percent activity included between any two of these values, at temperatures ranging from about 50° C. to about 110° C. For example, the enzyme or enzyme mixture demonstrates at least about 30%, 35%, 40%, 45%, 50%, 55%, 60%, 65%, or 75% of its maximum activity, or any percent activity included between any two of these values, at temperature conditions of about 50° C., 55° C., 60° C., 65° C., 70° C., 75° C., 80° C., 85° C., 90° C., 95° C., 100° C., 105° C., 110° C., or any temperature included between any two of these values.

In some embodiments, the enzyme or enzyme mixture demonstrates loss of enzymatic activity at ambient temperature and neutral pH ranges. For example, hyperthermophilic enzymes can undergo loss of activity at temperatures ranging from about 25° C. to 45° C., or from about 30° C. to 37° C. Acidophilic enzymes can undergo loss of activity at neutral pH values of from about 4.5 to 7.0 or above. In embodiments where a hyperthermophilic and/or acidophilic enzyme is provided, lowering temperature conditions to 25° C. to 45° C., and/or raising pH conditions to about 4.5 or above, can result in loss of enzymatic activity. In some embodiments, lowering temperature conditions to about 30° C. to 37° C., and/or raising pH conditions to about 4.5 to 7.0, can result in loss of enzymatic activity. In some embodiments, lowering temperature conditions to about 30° C. to 37° C., and/or raising pH conditions to about 7.0 or above, can result in loss of enzymatic activity. In some embodiments, lowering temperature conditions to about 25° C. to 45° C., or to about 30° C. to 37° C., is sufficient to result in loss of enzymatic activity. In some embodiments, raising the pH to about 4.5 or above, or to about 4.5 to 7.0, or to about 7.0 and above, is sufficient to result in loss of enzymatic activity. In some embodiments, lowering temperature conditions to about 25° C. to 45° C. and raising pH conditions to about 4.5 or above results in loss of enzymatic activity. In some embodiments, lowering temperature conditions to about 30° C. to 37° C. and raising pH conditions to about 4.5 to 7.0 results in loss of enzymatic activity. In some embodiments, lowering temperature conditions to about 30° C. to 37° C. and raising pH conditions to about 7.0 or above results in loss of enzymatic activity. Loss of enzymatic activity can mean a reduction of at least about 25%, 30%, 35%, 40%, 45%, 50%, 55%, 60%, 65%, 70%, 75%, 80%, 85%, 90%, 95% or 100% of enzymatic activity relative to baseline levels at non-ambient temperatures (e.g., about 50° C. to 110° C.) and non-neutral (e.g., about 0.5 to 4.5) pH ranges.

The enzyme or enzymes provided in the composition can be a protease, a lipase, a cellulase, a hemicellulase, a glycoside hydrolase, an endoprotease, a carboxyesterase, an amylase, an alpha-amylase, an endoglucanase, an endopullulanase, a PNGase, a b-glycosidease, a trehalase, a pullulanase, a peptidase, a signal peptidase, a xylanase, a cellobiohydrolase (CBH), a β-glucosidase, a peroxidase, a phospholipase, an esterase, a cutinase, a pectinase, a pectate lyase, a mannanase, a keratinase, a reductase, an oxidase, a phenoloxidase, a lipoxygenase, a ligninase, a tannase, a pentosanase, a malanase, a β-glucanase, an arabinosidase, a hyaluronidase, a chondroitinase, a laccase, a xyloglucanase, a xanthanase, an acyltransferase, a galactanase, a xanthan lyase, a xylanase, an arabinase, and combinations thereof.

In some embodiments, the enzyme is one that is isolated from a hyperthermophilic or thermophilic organism. In some embodiments, the enzyme is one that is isolated from an acidophilic organism. Exemplary organisms from which suitable enzymes can be isolated include, but are not limited to, an organism of the Archaea domain, the Bacteria domain or the Fungi domain. In some embodiments, the enzyme is isolated from an Archaea organism that is hyperthermophilic and/or acidophilic. For example, enzymes can be isolated from an organism of the Sulfolobales order, the Thermococcales order, the Thermoproteales order, the Acidilobales order, the Thermoplasmatales order, and the like. In some embodiments, the enzyme is isolated from a bacteria that is hyperthermophilic and/or acidophilic. For example, enzymes can be isolated from an organism of the Actinomycetales order, the Thermales order, the Thermoanaerobacteriales order, the Clostridiales order, the Acidothiobacillales order, the Nitrospirales order, the Rhodospirillales order, and the like. In some embodiments, the enzyme is isolated from a fungi that is hyperthermophilic and/or acidophilic.

In some embodiments, the enzyme is one that can be identified and isolated as described in WO 2014/081973. Enzymes having sequences as described in WO 2014/081973 can also be suitable for use in the compositions disclosed herein. For example, protease enzymes having amino acid sequences as described in WO 2014/081973 (e.g., SEQ ID NOs: 25-35) can be incorporated into the compositions disclosed herein.

### Additives

At least one additive can also be employed for the compositions disclosed herein. For example, an acid may be added in order to reduce the pH to a desired pH range. Suitable acids for use in the compositions include, for example, nitric acid, phosphoric acid, hydrofluoric acid, sulfuric acid, hydrochloric acid, acetic acid, paracetic acid, peroxyacetic acid, citric acid, glycolic acid, lactic acid, formic acid, methane sulfonic acid, alkyl C₈₋₁₀ polyglycolic acid, and mixtures or combinations thereof. The acid can be added in any amount ranging from about 0.1 wt % to 85 wt %, or from about 0.5 wt % to 80 wt %, or from about 1 wt % to about 75 wt %, or from about 2.5 wt % to about 70 wt %, or from about 5 wt % to about 65 wt %, or from about 10 wt % to about 60 wt %, or from about 15 wt % to about 55 wt %, or from about 20 wt % to about 50 wt %, or from about 25 wt % to about 45 wt %, or from about 30 wt % to 40 wt %, or any range included between and including any two of these values. For example, the amount of acid can be about 0.1 wt %, 0.25 wt%, 0.5 wt %, 1 wt %, 2.5 wt %, 5 wt %, 7.5 wt %, 10 wt %, 12.5 wt %, 15 wt %, 17.5 wt %, 20 wt %, 25 wt %, 30 wt %, 35 wt %, 40 wt %, 45 wt %, 50 wt %, 55 wt %, 60 wt %, 65 wt %, 70 wt %, 75 wt %, 80 wt %, 85 wt %, or any amount included between any two of these values.

In some embodiments, where mixtures or combinations of two or more acids are provided, the total amount of acid can range from about 0.1 wt % to 85 wt %, or from about 0.5 wt % to 80 wt %, or from about 1 wt % to about 75 wt %, or from about 2.5 wt % to about 70 wt %, or from about 5 wt % to about 65 wt %, or from about 10 wt % to about 60 wt %, or from about 15 wt % to about 55 wt %, or from about 20 wt % to about 50 wt %, or from about 25 wt % to about 45 wt %, or from about 30 wt % to 40 wt %, or any range included between and including any two of these values. For example, the total amount of acid can be about 0.1 wt %, 0.5 wt %, 1 wt %, 2.5, wt %, 5 wt %, 7.5 wt %, 10 wt %, 12.5 wt %, 15 wt %, 17.5 wt %, 20 wt %, 25 wt %, 30 wt %, 35 wt %, 40 wt %, 45 wt %, 50 wt %, 55 wt %, 60 wt %, 65 wt %, 70 wt %, 75 wt %, 80 wt %, 85 wt %, or any amount included between any two of these values. In an exemplary embodiment, the composition can contain about 45% nitric acid and 5% phosphoric acid.

Other additives can also be provided to the composition. Exemplary additives used in industrial cleaning and sanitation protocols in the dairy industry include, for example, Bright Cleanse No. 321 (Hydrite Chemical Co., Product No. FP032101), DetBuild No. 394 (Hydrite Chemical Co., Product No. FP039401), PerasanA (Enviro Tech Chemical Services, Inc.), MPA No. 168 (Hydrite Chemical Co., Product No. FP016801), and the like. In some embodiments, a suitable additive can be at least one selected from the group consisting of: poly(oxy-1,2-ethanediyl), alpha-(nonylphenyl)-omega-hydroxy-, dipropylene glycol monomethyl ether, sodium xylene sulfonate, potassium 4-dodecylbenzene sulfonate, triethanolamine dodecylbenzene sulfonate, triethanolamine, hydrogen peroxide, D-glucopyranose (oligomeric, decy octyl glycosides), D-glucopyranose (oligomeric, C₁₀₋₁₆-alkyl glycosides), sodium formate, sodium hydroxide, tetrasodium EDTA, and water.

In some embodiments, the additive can comprise a solvent such as, for example, an alkanol or a polyol. The alkanol can be soluble or miscible with water and lipids, and comprises a C₁ to C₁₀ alkyl group that is straight or branched, substituted or non-substituted. Useful alkanols include short chain alcohols, such as C₁-C₈ primary, secondary and tertiary alcohols, e.g., methanol, ethanol, n-propanol, iso-propanol, and butanol. Exemplary alkanols include the various isomers of C₃ alcohols, particularly iso-propanol. C₁-C₈ diols can also be used in the alkanol constituent.

The polyol can be an alkylene glycol, such as, for example, glycerol, ethylene glycol, propylene glycol, 1,2-propylene glycol, 1,3-propylene glycol, glycerine, 1,4-butylene glycol and mixtures thereof.

In some embodiments, the additive comprises an anti-foam component, such as, for example, a silicone-based anti-foam component.

In some embodiments, the additive includes an alkanolamine selected from the group consisting of: monoalkanolamine, dialkanolamine, trialkanolamine, alkylalkanolamine, trialkylamine, triethanolamine and combinations thereof.

In some embodiments, the additive includes a conventional enzyme stabilizing agent, e.g. a polyol such as propylene glycol or glycerol, a sugar or sugar alcohol, lactic acid, boric acid, or a boric acid derivative, e.g. an aromatic borate ester, or a phenyl boronic acid derivative such as 4-formylphenyl boronic acid.

In some embodiments, additive includes one or more food grade agents. The food grade agent can be selected from the group consisting of: hydroxide, carbonate, bicarbonate, silicate (SiO₄⁴⁻), monoethanolamine, peroxy acid, hydrogen peroxide, an ethoxylated alcohol, an alkylpolyglycoside, ethyleneoxide/propylene oxide copolymer, octenylsuccinic anhydride, octenylsuccinic acid, aminotrimethylene phosphonic acid, phosphono-1,2,4-butanetricaboxylic acid, gluconic acid, a maleic acid/olefin-copolymer, polyacrylic acid, ethylene diamine tetraacetic acid (EDTA), glutamic acid diacetic acid (GLDA), methyl glycine diacetic acid (MGDA), nitrilo triacetic acid (NTA), alkyl (C₈₋₂₄) dibasic fatty acid, tripolyphosphoric acid, hexametaphosphoric acid, caprylic acid, sorbic acid, polyalkylene glycol, lauryl dimethyl betaine, and a polydimethyl siloxane emulsion. In some embodiments, the one or more food grade agents are GRAS Certified. In some embodiments, the one or more food grade agents are GRAS compliant (i.e., the agent or substance is not registered but would qualify for registration as a food grade additive).

In some embodiments, the additive includes a chelating agent. The chelating agent can be, for example, a metal ion chelating agent. Metal ion chelating agents can include, for example, copper, iron and/or manganese chelating agents and mixtures thereof. Such chelating agents can be selected from the group consisting of: phosphonates, amino carboxylates, amino phosphonates, succinates, polyfunctionally-substituted aromatic chelating agents, 2-pyridinol-N-oxide compounds, hydroxamic acids, carboxymethyl inulins and mixtures thereof. Chelating agents can be present in the acid or salt form including alkali metal, ammonium, and substituted ammonium salts thereof, and mixtures thereof.

Aminocarboxylates chelating agents include, but are not limited to, ethylenediaminetetracetates (EDTA); ethylene glycol tetraacetates (EGTA), N-(hydroxyethyl)ethylenediaminetriacetates (HEDTA); nitrilotriacetates (NTA); ethylenediamine tetraproprionates; triethylenetetraaminehexacetates, diethylenetriamine-pentaacetates (DTPA); methylglycinediacetic acid (MGDA); Glutamic acid diacetic acid (GLDA); ethanoldiglycines; triethylenetetraaminehexaacetic acid (TTHA); N-hydroxyethyliminodiacetic acid (HEIDA); dihydroxyethylglycine (DHEG); ethylenediaminetetrapropionic acid (EDTP), trans-1,2-diamino-cyclohexan-N,N,N′,N′-tetraacetic acid (CDTA), nitrilo-2,2′,2″-triacetic acid, diethylenetriamine-N,N,N′,N′,N″-pentaacetic acid, methylamine, histidine, malate and phytochelatin, hemoglobin, chlorophyll, siderophore, pyocyanin, pyoverdin, Enterobactin, peptides and sugars, humic acid, citric acid, water softeners, phosphonates, tetracycline, gadolinium, organophosphorus compound 2,2′-bis(diphenylphosphino)-1,1′-binaphthyl, pentetic acid; N,N-Bis(2-(bis-(carboxymethyl)amino)ethyl)-glycine, N,N-bis(carboxymethyl)glycine, triglycollamic acid; [(Carboxymethyl)imino]bis-(ethylenenitrilo)]-tetraacetic acid), Trilone A, α, α′, α″-trimethylaminetricarboxylic acid, tri(carboxymethyl)amine, aminotriacetic acid, Titriplex i, and Hampshire NTA acid, and salts and derivatives thereof.

Phosphorus-containing chelating agents include, but are not limited to, diethylene triamine penta (methylene phosphonic acid) (DTPMP CAS 15827-60-8); ethylene diamine tetra(methylene phosphonic acid) (EDTMP CAS 1429-50-1); 2-Phosphonobutane 1,2,4-tricarboxylic acid (Bayhibit® AM); hexamethylene diamine tetra(methylene phosphonic acid) (CAS 56744-47-9); hydroxy-ethane diphosphonic acid (HEDP CAS 2809-21-4); hydroxyethane dimethylene phosphonic acid; 2-phosphono-1,2,4-Butanetricarboxylic acid (CAS 37971-36-1); 2-hydroxy-2-phosphono-Acetic acid (CAS 23783-26-8); Aminotri(methylenephosphonic acid) (ATMP CAS 6419-19-8); P,P′-(1,2-ethanediyl)bis-Phosphonic acid (CAS 6145-31-9); P,P′-methylenebis-Phosphonic acid (CAS 1984-15-2); Triethylenediaminetetra(methylene phosphonic acid) (CAS 28444-52-2); P-(1-hydroxy-1-methylethyl)-Phosphonic acid (CAS 4167-10-6); bis(hexamethylene triamine penta(methylenephosphonic acid)) (CAS 34690-00-1); N2,N2,N6,N6-tetrakis(phosphonomethyl)-Lysine (CAS 194933-56-7, CAS 172780-03-9), salts thereof, and mixtures thereof. Preferably, these aminophosphonates do not contain alkyl or alkenyl groups with more than about 6 carbon atoms.

A biodegradable chelator that can also be used herein is ethylenediamine disuccinate (EDDS). In some embodiments, the [S,S] isomer as described in U.S. Pat. No. 4,704,233 can be used. In some embodiments, the trisodium salt of EDDA can be used, though other forms, such as magnesium salts, are also be useful. Polymeric chelating agents such as Triton P® can also be useful.

Polyfunctionally-substituted aromatic chelating agents can also be used in the compositions disclosed herein. Compounds of this type in acid form are dihydroxydisulfobenzenes, such as 1,2-dihydroxy-3,5-disulfobenzene, also known as Tiron. Other sulphonated catechols may also be used. In addition to the disulfonic acid, the term “tiron” can also include mono- or di-sulfonate salts of the acid, such as, for example, the disodium sulfonate salt, which shares the same core molecular structure with the disulfonic acid.

The chelating agent can also include a substituted or unsubstituted 2-pyridinol-N-oxide compound or a salt thereof, can also be provided as a chelating agent. This includes tautomers of the compound, e.g., 1-Hydroxy-2(1H)-pyridinone, as a chelating agent. In some embodiments, the chelating agent is selected from the group consisting of: 2-hydroxypyridine-1-oxide; 3-pyridinecarboxylic acid, 2-hydroxy-, 1-oxide; 6-hydroxy-3-pyridinecarboxylic acid, 1-oxide; 2-hydroxy-4-pyridinecarboxylic acid, 1-oxide; 2-pyridinecarboxylic acid, 6-hydroxy-, 1-oxide; 6-hydroxy-3-pyridinesulfonic acid, 1-oxide; and mixtures thereof. In some embodiments, the 1-Hydroxy-2(1H)-pyridinone compound is selected from the group consisting of: 1-Hydroxy-2(1H)-pyridinone (CAS 822-89-9); 1,6-dihydro-1-hydroxy-6-oxo-3-Pyridinecarboxylic acid (CAS 677763-18-7); 1,2-dihydro-1-hydroxy-2-oxo-4-Pyridinecarboxylic acid (CAS 119736-22-0); 1,6-dihydro-1-hydroxy-6-oxo-2-Pyridinecarboxylic acid (CAS 94781-89-2); 1-hydroxy-4-methyl-6-(2,4,4-trimethylpentyl)-2(1H)-Pyridinone (CAS 50650-76-5); 6-(cyclohexylmethyl)-1-hydroxy-4-methyl-2(1H)-Pyridinone (CAS 29342-10-7); 1-hydroxy-4,6-dimethyl-2(1H)-Pyridinone (CAS 29342-02-7); 1-Hydroxy-4-methyl-6-(2,4,4-trimethylpentyl)-2-pyridone monoethanolamine (CAS 68890-66-4); 1-hydroxy-6-(octyloxy)-2(1H)-Pyridinone (CAS 162912-64-3); 1-Hydroxy-4-methyl-6-cyclohexyl-2-pyridinone ethanolamine salt (CAS 41621-49-2); 1-Hydroxy-4-methyl-6-cyclohexyl-2-pyridinone (CAS 29342-05-0); 6-ethoxy-1,2-dihydro-1-hydroxy-2-oxo-4-Pyridinecarboxylic acid, methyl ester (CAS 36979-78-9); 1-hydroxy-5-nitro-2(1H)-Pyridinone (CAS 45939-70-6); and mixtures thereof.

Chelating agents can also include hydroxamic acids, which are a class of chemical compounds in which a hydroxylamine is inserted into a carboxylic acid. The general structure of a hydroxamic acid is the following:

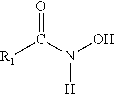

Suitable hydroxamates are those where R₁ is C₄- to C₁₄-alkyl, including normal alkyl, saturated alkyl, salts thereof and mixtures thereof. For example, when the C₈-alkyl is present, the compound is called octyl hydroxamic acid.

In some embodiments, the additive can be a stabilizer, such as, for example, a hyaluronic acid stabilizer, a polyvinylpyrrolidone stabilizer, or a polyol stabilizer. Exemplary polyols are disclosed herein and include, for example, propylene glycol and glycerol. In some embodiments, the stabilizer is albumin or a sugar or sugar alcohol, such as, for example, mannitol or sorbitol. In some embodiments, the stabilizer is a salt, such as, for example, potassium chloride, magnesium sulfate, and the like. In some embodiments, the stabilizer is an enzyme stabilizer. Any conventional enzyme stabilizer can be used, for example, water-soluble sources of calcium and/or magnesium ions. In some embodiments, the enzyme stabilizer can be a reversible protease inhibitor, such as, for example, a lactic acid or a boron compound. Exemplary boron compounds include, but are not limited to, borate, 4-formyl phenylboronic acid, phenylboronic acid and derivatives thereof. In some embodiments, the enzyme stabilizer can be, but is not limited to, compounds such as calcium formate, sodium formate and 1,2-propane diol.

The additive can be provided in the composition in any amount ranging from about 0.05 wt % to 85 wt %, or from about 0.1 wt % to 80 wt %, or from about 0.5 wt % to about 75 wt %, or from about 1 wt % to about 70 wt %, or from about 2.5 wt % to about 65 wt %, or from about 5 wt % to about 60 wt %, or from about 10 wt % to about 55 wt %, or from about 15 wt % to about 50 wt %, or from about 20 wt % to about 45 wt %, or from about 25 wt % to 40 wt %, or any range included between and including any two of these values. For example, the amount of additive provided in the composition can be about 0.05 wt%, 0.1 wt %, 0.25%, 0.5 wt %, 1 wt %, 2.5, wt %, 5 wt %, 7.5 wt %, 10 wt %, 12.5 wt %, 15 wt %, 17.5 wt %, 20 wt %, 25 wt %, 30 wt %, 35 wt %, 40 wt %, 45 wt %, 50 wt %, 55 wt %, 60 wt %, 65 wt %, 70 wt %, 75 wt %, 80 wt %, 85 wt %, or any amount included between any two of these values.

The composition can include one or more surfactants, which may be an anionic surfactant, a cationic surfactant, a non-ionic surfactant, a semi-polar surfactant, a zwitterionic surfactant, a fatty acid type surfactant, a modified fatty acid surfactant, a polysorbate, an amphoteric surfactant, a polysaccharide surfactant, a silicone emulsion, a hydrotrope, or a mixture thereof.

Exemplary anionic surfactants that can be provided in the compositions disclosed herein include, but are not limited to, sulfates and sulfonates, e.g., linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or soap, and combinations thereof.

Exemplary cationic surfactants that can be provided in the compositions disclosed herein include, but are not limited to, alklydimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, and combinations thereof.

Exemplary non-ionic surfactants that can be provided in the compositions disclosed herein include, but are not limited to, alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxy alkyl fatty acid amides, or N-acyl N-alkyl derivatives of glucosamine (glucamides, GA, or fatty acid glucamide, FAGA), as well as products available under the trade names SPAN® and TWEEN®, the ethoxylates of alkyl polyethylene glycol ethers, polyalkylene glycol (e.g., 100% Breox FCC92) and alcohol alkoxylate EO/PO (e.g., Plurafac LF403). Exemplary alcohol ethoxylates include fatty alcohol ethoxylates, e.g., tridecyl alcohol alkoxylate, ethylene oxide adduct, alkyl phenol ethoxylates, and ethoxy/propoxy block surfactants, and combinations thereof.

Exemplary semipolar surfactants that can be provided in the compositions disclosed herein include, but are not limited to, amine oxides (AO) such as alkyldimethylamineoxide, N-(coco alkyl)-N,N-dimethylamine oxide and N-(tallow-alkyl)-N,N-bis(2-hydroxyethyl)amine oxide, fatty acid alkanolamides and ethoxylated fatty acid alkanolamides, and combinations thereof.

Exemplary zwitterionic surfactants that can be provided in the compositions disclosed herein include, but are not limited to, betaine, alkyldimethylbetaine, sulfobetaine, and combinations thereof.

Further non-limiting examples of a surfactant include a fatty acid type surfactant such as caprylic acid (e.g., 100% Prifrac 2912). Non-limiting examples of a modified fatty acid include, e.g., alkyl (C₂₁) dibasic fatty acid, Na salt (40%, Diacid H240). Non-limiting examples of a polysorbate include potassium sorbate (e.g., Tween® 20/60/80). Non-limiting examples of an amphoteric surfactant include lauryl dimethyl betaine (e.g., Empigen BB). Non-limiting examples of a polysaccharide surfactant include alkyl C₈-C₁₀ polyglycoside (e.g., 70% Triton® BG10). Non-limiting examples of a silicone emulsion include a polydimethyl siloxane emulsion (e.g., Dow Corning Antifoam 1510).

A hydrotrope is a compound that dissolves hydrophobic compounds in aqueous solutions. Typically, hydrotropes consist of a hydrophilic part and a hydrophobic part (similar to surfactants) but the hydrophobic part is generally too small to cause spontaneous self aggregation. Exemplary hydrotropes include, but are not limited to, benzene sulfonates, naphthalene sulfonates, alkyl benzene sulfonates, naphthalene sulfonates, alkyl sulfonates, alkyl sulfates, alkyl diphenyloxide disulfonates, and phosphate ester hydrotropes. Exemplary alkyl benzene sulfonates include, for example, isopropylbenzene sulfonates, xylene sulfonates, toluene sulfonates, cumene sulfonates, as well as mixtures any two or more thereof. Exemplary alkyl sulfonates include hexyl sulfonates, octyl sulfonates, and hexyl/octyl sulfonates, and mixtures of any two or more thereof.

Additional exemplary surfactants include, but are not limited to, CHAPS, Pluronic® F-68, NP-40, sodium dodecyl sulfate (SDS), polysorbate 20, a saponin, Triton® X-100, sarkosyl, and mixtures of combinations thereof.

The surfactant can be provided in the composition in any amount ranging from about 0.05 wt % to 85 wt %, or from about 0.1 wt % to 80 wt %, or from about 0.5 wt % to about 75 wt %, or from about 1 wt % to about 70 wt %, or from about 2.5 wt % to about 65 wt %, or from about 5 wt % to about 60 wt %, or from about 10 wt % to about 55 wt %, or from about 15 wt % to about 50 wt %, or from about 20 wt % to about 45 wt %, or from about 25 wt % to 40 wt %, or any range included between and including any two of these values. For example, the amount of surfactant provided in the composition can be about 0.05 wt%, 0.1 wt %, 0.25 wt%, 0.5 wt %, 1 wt %, 2.5, wt %, 5 wt %, 7.5 wt %, 10 wt %, 12.5 wt %, 15 wt %, 17.5 wt %, 20 wt %, 25 wt %, 30 wt %, 35 wt %, 40 wt %, 45 wt %, 50 wt %, 55 wt %, 60 wt %, 65 wt %, 70 wt %, 75 wt %, 80 wt %, 85 wt %, or any amount included between any two of these values.

In embodiments wherein two or more surfactants are provided in the composition, the total amount of surfactant in the composition can be any amount ranging from about 0.05 wt % to 85 wt %, or from about 0.1 wt % to 80 wt %, or from about 0.5 wt % to about 75 wt %, or from about 1 wt % to about 70 wt %, or from about 2.5 wt % to about 65 wt %, or from about 5 wt % to about 60 wt %, or from about 10 wt % to about 55 wt %, or from about 15 wt % to about 50 wt %, or from about 20 wt % to about 45 wt %, or from about 25 wt % to 40 wt %, or any range included between and including any two of these values. For example, the total amount of surfactant can be about 0.05 wt%, 0.1 wt %, 0.5 wt %, 1 wt %, 2.5, wt %, 5 wt %, 7.5 wt %, 10 wt %, 12.5 wt %, 15 wt %, 17.5 wt %, 20 wt %, 25 wt %, 30 wt %, 35 wt %, 40 wt %, 45 wt %, 50 wt %, 55 wt %, 60 wt %, 65 wt %, 70 wt %, 75 wt %, 80 wt %, 85 wt %, or any amount included between any two of these values.

### Methods of Cleaning

Also provided herein are methods of cleaning a surface, wherein the method comprises: providing a cleaning composition as disclosed herein, and contacting a soiled surface with the composition, such that at least a portion of the soil is removed from the soiled surface; wherein the temperature of the composition ranges from about 50° C. to about 110° C. and has a pH of from about 0.5 to about 7.0.

The surface can be soiled with at least one foreign substance selected from the group consisting of: a residue of a grain, a dairy product, an alcoholic beverage, a non-alcoholic beverage, a fruit, a vegetable, a meat, an animal food, a soiled dish residue, an industrial fermentation product, an algae, a biofuel, a pharmaceutical, a nutritional supplement, a cosmetic or a combination of any two or more thereof. In some embodiments, the surface is soiled with a contaminating protein, a sugar, a fat or fatty acid, or combinations thereof. For example, the surface can be a membrane that is fouled with aggregated milk proteins.

In some embodiments, the temperature of the composition ranges from about 50° C. to about 110° C. In some embodiments, the temperature of the composition is about 50° C., 55° C., 60° C., 65° C., 70° C., 75° C., 80° C., 85° C., 90° C., 95° C., 100° C., 105° C., 110° C., or any temperature included between any two of these values.

In some embodiments, the composition has a pH of from about 0.5 to about 4.5, or from about 0.5 to about 3.0, or from about 0.5 to about 1.5, or any pH range included between and including any two of these values. In some embodiments, the composition has a pH of about 2.0 to 3.0. In some embodiments, the composition has a pH of from about 4 to about 7, or from about 4.5 to about 6.5, or from about 5 to about 6, or any pH range included between and including any two of these values. In some embodiments, the composition has a pH of about 5.5. In some embodiments, the composition has a pH of about 3.0.

In some embodiments, the method results in at least about 10%, 15%, 20%, 25%, 30%, 35%, 40%, 45%, 50%, 55%, 60%, 65%, 70%, 75%, 80%, 85%, 90%, 95% of soil removal from the surface. In some embodiments, the method results in at least about 10% of soil removal from the surface. In some embodiments, the method results in at least about 15% of soil removal from the surface. In some embodiments, the method results in at least about 20% of soil removal from the surface. In some embodiments, the method results in at least about 25% of soil removal from the surface. In some embodiments, the method results in at least about 30% of soil removal from the surface. In some embodiments, the method results in at least about 35% of soil removal from the surface. In some embodiments, the method results in at least about 40% of soil removal from the surface. In some embodiments, the method results in at least about 45% of soil removal from the surface. In some embodiments, the method results in at least about 50% of soil removal from the surface. In some embodiments, the method results in at least about 55% of soil removal from the surface. In some embodiments, the method results in at least about 60% of soil removal from the surface. In some embodiments, the method results in at least about 65% of soil removal from the surface. In some embodiments, the method results in at least about 70% of soil removal from the surface. In some embodiments, the method results in at least about 75% of soil removal from the surface. In some embodiments, the method results in at least about 80% of soil removal from the surface. In some embodiments, the method results in at least about 85% of soil removal from the surface. In some embodiments, the method results in at least about 90% of soil removal from the surface. In some embodiments, the method results in at least about 95% of soil removal from the surface. In some embodiments, the method results in about 100% of soil removal, or total soil removal, from the surface.

Contact between the soiled surface and the composition can be for any duration of time ranging from about 5 minutes to about 180 minutes, or from about 10 minutes to about 150 minutes, or from about 15 minutes to about 120 minutes, or from about 20 minutes to about 90 minutes, or from about 30 minutes to about 75 minutes, or from about 40 minutes to about 60 minutes, or any range included between and including any two of these values. In some embodiments, the surface is in contact with the composition for at least about 120 minutes. In some embodiments, the surface is in contact with the composition for at least about 90 minutes. In some embodiments, the surface is in contact with the composition for at least about 60 minutes. In some embodiments, the surface is in contact with the composition for at least about 45 minutes. In some embodiments, the surface is in contact with the composition for at least about 30 minutes. In some embodiments, the surface is in contact with the composition for at least about 20 minutes. In some embodiments, the surface is in contact with the composition for at least about 10 minutes. In some embodiments, the surface is in contact with the composition for at least about 5 minutes.

In some embodiments, the surface to be cleaned is in contact with the composition for less than about 5 minutes. For example, the surface can be in contact with the composition for about 2 seconds, about 5 seconds, about 10 seconds, about 15 seconds, about 20 seconds, about 30 seconds, about 60 seconds, about 90 seconds, about 120 seconds, for about 3 minutes, or for about 4 minutes.

In some embodiments, the method further comprises adding a sufficient amount of aqueous solution or water to decrease the temperature and/or raise the pH of the composition. In some embodiments, the temperature of the composition is decreased to about 30° C. to 37° C. In some embodiments, the pH of the composition rises to a range of 4.5 to 7.0. In some embodiments, the addition of aqueous solution or water decreases the cleaning activity of the composition. In some embodiments, the addition of aqueous solution or water reduces the enzymatic activity of an enzyme in the composition.

In some embodiments, the method further comprises rinsing the surface with an aqueous solution or water, wherein the rinsing results in a decrease in temperature and a rise in pH conditions. In some embodiments, the rinse step results in a decrease in temperature to about 25° C. to 45° C., or to about 30° C. to 37° C. In some embodiments, the rinse step results in a rise in pH to a range of 4.5 to 7.0 or above. In some embodiments, the rinse step results in a decrease in the cleaning activity of the composition. In some embodiments, the rinse step results in a reduction in enzymatic activity of any enzyme remaining on the surface.

In some embodiments, the rinse step results in a decrease in the cleaning activity of the composition. For example, the rinse step can result in a decrease by about 25%, 30%, 35%, 40%, 45%, 50%, 55%, 60%, 65%, 70%, 75%, 80%, 85%, 90%, 95% or 100% of cleaning activity relative to baseline (e.g., activity prior to the rinse step). In some embodiments, the rinse step can result in a decrease of at least about 25%, 30%, 35%, 40%, 45%, 50%, 55%, 60%, 65%, 70%, 75%, 80%, 85%, 90%, 95%, 98% or 99% of cleaning activity relative to baseline. In some embodiments, the rinse step can result in a 100% decrease of cleaning activity relative to baseline. In some embodiments, the rinse step can result in complete elimination of cleaning activity relative to baseline.

In some embodiments, the rinse step results in a reduction in enzymatic activity of the composition. For example, the rinse step can result in a decrease by about 25%, 30%, 35%, 40%, 45%, 50%, 55%, 60%, 65%, 70%, 75%, 80%, 85%, 90%, 95% or 100% of enzymatic activity relative to baseline (e.g., enzymatic activity prior to the rinse step). In some embodiments, the rinse step can result in a decrease of at least about 25%, 30%, 35%, 40%, 45%, 50%, 55%, 60%, 65%, 70%, 75%, 80%, 85%, 90%, 95%, 98% or 99% of enzymatic activity relative to baseline. In some embodiments, the rinse step can result in a 100% decrease of enzymatic activity relative to baseline. In some embodiments, the rinse step can result in complete elimination of enzymatic activity relative to baseline.

In some embodiments, the rinse step results in a reduction in enzymatic activity of any enzyme remaining on the surface being cleaned. For example, the rinse step can result in a decrease by about 25%, 30%, 35%, 40%, 45%, 50%, 55%, 60%, 65%, 70%, 75%, 80%, 85%, 90%, 95% or 100% of enzymatic activity relative to baseline (e.g., enzymatic activity prior to the rinse step). In some embodiments, the rinse step can result in a decrease of at least about 25%, 30%, 35%, 40%, 45%, 50%, 55%, 60%, 65%, 70%, 75%, 80%, 85%, 90%, 95%, 98% or 99% of enzymatic activity relative to baseline. In some embodiments, the rinse step can result in a 100% decrease of enzymatic activity relative to baseline. In some embodiments, the rinse step can result in complete elimination of enzymatic activity relative to baseline.

In some embodiments, the composition solution is applied to soiled industrial equipment to clean the industrial equipment.

In some embodiments, the method of cleaning comprises mechanical dish-washing. In some embodiments, the surface is placed in a dish-washing apparatus (e.g., dishwasher) prior to application of the composition to the surface. The dishwasher can be used to clean cooking and eating articles, such as, e.g., dishes, bowls, cups, glasses, pots, pans, utensils and other cooking or food-serving equipment.

In some embodiments, the method of cleaning comprises a clean-in-place (CIP) or a clean-out-of-place (COP) method. CIP systems include the internal components of industrial equipment such as tanks, lines, pumps and other equipment used for processing typically liquid product streams such as beverages, milk, and juices. COP systems include readily accessible vessels of industrial equipment, including wash tanks, soaking vessels, holding tanks, scrub sinks, vehicle parts washers, noncontinuous batch washers and systems, and the like.

Automated clean-in-place (CIP) techniques have reduced the need for industrial equipment disassembly and increased the efficiency of cleaning and sanitizing methods. CIP techniques use the combination of chemistry and mechanical action to clean the inside of industrial equipment without requiring the time consuming and labor intensive disassembly and manual cleaning of a system. CIP techniques generally include the circulation of chemistries (e.g., cleaners) for periodic cleaning of industrial equipment. In some embodiments, CIP techniques involve a first rinse, the application of cleaning solutions, a second rinse with potable water, followed by resumed operations. In some embodiments, one or both rinses are omitted. The process can also include any other contacting step in which a rinse, acidic or basic functional fluid, solvent or other cleaning component such as hot water, cold water, etc. can be contacted with the equipment at any step during the process.

Industrial equipment (e.g., brewery equipment or dairy equipment) can be cleaned using CIP techniques. The cleaning of the in-place systems can be accomplished with the compositions disclosed herein, and according to the present methods. In some embodiments, the compositions are heated prior to introduction into the in-place systems. For example, the compositions can be heated to about 50° C., 55° C., 60° C., 65° C., 70° C., 75° C., 80° C., 85° C., 90° C., 95° C., 100° C., 105° C., 110° C., or any temperature included between any two of these values. In some embodiments, a concentrated composition can be introduced into an in-place system (e.g., industrial equipment) and diluted, in situ, with water. In some embodiments, the composition is applied or introduced into the system as a use-solution. CIP techniques typically employ flow rates of about 0.1 meters per second to about 0.5 meters per second, about 1.0 meter per second, about 1.1 meters per second, about 1.2 meters per second, about 1.3 meters per second, about 1.4 meters per second, about 1.5 meters per second, about 1.6 meters per second, about 1.7 meters per second, about 1.8 meters per second, about 1.9 meters per second, about 2.0 meters per second, about 2.5 meters per second, about 3.0 meters per second, about 3.5 meters per second, about 4.0 meters per second, about 4.5 meters per second, about 5.0 meters per second or a range between and including any two of these values.

CIP techniques can employ the compositions disclosed herein with heated (e.g., about 50° C., 60° C., 70° C., 80° C., 85° C., 90° C., 95° C., 100° C., 105° C., 110° C.) water. CIP techniques can employ contact times of at least about 2 seconds, about 5 seconds, about 10 seconds, about 15 seconds, about 20 seconds, about 30 seconds, about 60 seconds, about 90 seconds, about 120 seconds, 5 minutes, 15 minutes, 30 minutes, one hour, two hours, or a range between and including any two of these values.

In some embodiments, the method of cleaning further comprises collecting the composition as an effluent composition subsequent to contacting the soiled surface with the composition. Once the surface has been cleaned, and the soiled residues have dissolved or become suspended in the composition, the effluent composition is recovered. In some embodiments, the effluent composition is optionally concentrated by removal of liquid from the composition. In some embodiments, the effluent composition is filtered to remove any soil that is not in solution.

In some embodiments, the effluent composition, whether directly recovered after cleaning the surface or optionally concentrated after recovery, is stored in a holding vessel or container for from about 30 minutes to 8 days. For example, the effluent composition can be stored for at least 30 minutes, 1 hour, 2 hours, 4 hours, 6 hours, 8 hours, 12 hours, 18 hours, 24 hours, or for any duration of time between any two of these values. In some embodiments, the effluent composition can be stored for at least 1 day, 2 days, 3 days, 4 days, 5 days, 6 days, 7 days, or 8 days.

In some embodiments, the method of cleaning further comprises recycling or reusing the effluent composition, wherein the effluent composition is contacted with a second soiled surface. In some embodiments, the effluent composition is contacted with a second surface to be sanitized after being stored in a holding vessel or container for from about 30 minutes to 15 days. For example, the effluent composition can be contacted with a second surface to be sanitized after being stored for at least 30 minutes, 1 hour, 2 hours, 4 hours, 6 hours, 8 hours, 12 hours, 18 hours, 24 hours, or for any duration of time between any two of these values. In some embodiments, the effluent composition can be contacted with a second surface to be sanitized after being stored for at least 1 day, 2 days, 3 days, 4 days, 5 days, 6 days, 7 days, 8 days, 9 days, 10 days, 11 days, 12 days, 13 days, 14 days, or 15 days. In some embodiments, the effluent composition is contacted with a second soiled surface after being recovered from the cleaning of a first soiled surface. In some embodiments, the effluent composition is contacted with a second soiled surface within about 5 minutes, 10 minutes, 15 minutes, 20 minutes, 25 minutes or 30 minutes after being recovered from the cleaning of a first soiled surface. In some embodiments, the effluent composition is contacted with a second soiled surface within about 5 minutes or directly after being recovered from the cleaning of a first soiled surface.

In some embodiments, the effluent composition can be reused at least once, twice, three times, four times, five times, six times, seven times, eight times, nine times, or ten times on separate soiled surfaces. For example, the effluent composition can be reused at least once on a second soiled surface, or reused a second time on a third soiled surface, or reused a third time on a fourth soiled surface, and so on.

In some embodiments, at least one enzyme is added to the effluent composition prior to its contact or reuse for cleaning on the separate soiled surfaces. For example, at least one enzyme can be added to the effluent composition prior to its reuse and contact with a second soiled surface, or prior to its reuse and contact with a third soiled surface, or prior to its reuse and contact with a fourth soiled surface, and so on. In some embodiments, the at least one enzyme can be added to the effluent composition prior to each time it is reused for application on a soiled surface. In some embodiments, the at least one enzyme can be added to the effluent composition prior to every other time it is reused for application on a soiled surface. The at least one enzyme can be added in any amount as disclosed herein, for example, from about 0.0001 mg to 1000 mg of enzyme protein per 100 grams of soil on the surface, or from about 0.0001 wt % to 50 wt % of the effluent composition, or provided in an activity range of from about 0.0001 to 100 activity units.

### Methods of Sanitizing

Also provided herein are methods of sanitizing a surface, wherein the method comprises: providing a sanitizing composition as disclosed herein, and contacting a surface with the composition, such that at least about 95% of living microbes are eliminated and/or killed on the surface upon contact; wherein the temperature of the composition ranges from about 50° C. to about 110° C. and has a pH of from about 0.5 to about 7.0.

In some embodiments, the temperature of the composition ranges from about 50° C. to about 110° C. In some embodiments, the temperature of the composition is about 50° C., 55° C., 60° C., 65° C., 70° C., 75° C., 80° C., 85° C., 90° C., 95° C., 100° C., 105° C., 110° C., or any temperature included between any two of these values.

In some embodiments, the composition has a pH of from about 0.5 to about 4.5, or from about 0.5 to about 3.0, or from about 0.5 to about 1.5, or any pH range included between and including any two of these values. In some embodiments, the composition has a pH of about 2.0 to 3.0. In some embodiments, the composition has a pH of from about 4 to about 7, or from about 4.5 to about 6.5, or from about 5 to about 6, or any pH range included between and including any two of these values. In some embodiments, the composition has a pH of about 5.5. In some embodiments, the composition has a pH of about 3.0.

In some embodiments, at least about 97% of living organisms are eliminated and/or killed on the surface upon contact between the surface and the composition. In some embodiments, at least about 99% of living organisms are eliminated and/or killed on the surface upon contact between the surface and the composition. In some embodiments, at least about 99.5% of living organisms are eliminated and/or killed on the surface upon contact between the surface and the composition. In some embodiments, at least about 99.9% of living organisms are eliminated and/or killed on the surface upon contact between the surface and the composition. In some embodiments, at least about 99.99% of living organisms are eliminated and/or killed on the surface upon contact between the surface and the composition. In some embodiments, at least about 99.999% of living organisms are eliminated and/or killed on the surface upon contact between the surface and the composition.

Exemplary living organisms targeted for elimination include, but are not limited to, microbes such as Enterococcus faecium, Streptococcus mutans, a Staphylococcus species, a Campylobacter species, a Clostridium species, a Bacillus species, an Enterobacter species, Listeria monocytogenes, E. coli O157:H7, Legionella pneumophila, Pseudomonas, Helicobacter pylori, Campylobacter jejuni, Clostridium perfringens, Clostridium difficile, Escherichia coli, Staphylococcus aureus, Salmonella spp., Salmonella typhimurium, Bacillus proteus, Bacillus subtilis, Bacillus cereus, Shigella spp., Streptococcus, Vibrio cholerae, Vibrio parahaemolyticus, Vibrio vulnificus, Yersinia enterocolitica, Yersinia pseudotuberculosis, Coxiella burnetii, Brucella spp., Corynebacterium ulcerans, Sarcinae spp. and Plesiomonas shigelloides.

In some embodiments, the living organism targeted for elimination includes a yeast or a mold.

Contact between the surface to be sanitized and the composition can be for any duration of time ranging from about 5 minutes to about 180 minutes, or from about 10 minutes to about 150 minutes, or from about 15 minutes to about 120 minutes, or from about 20 minutes to about 90 minutes, or from about 30 minutes to about 75 minutes, or from about 40 minutes to about 60 minutes, or any range included between and including any two of these values. In some embodiments, the surface is in contact with the composition for at least about 120 minutes. In some embodiments, the surface is in contact with the composition for at least about 90 minutes. In some embodiments, the surface is in contact with the composition for at least about 60 minutes. In some embodiments, the surface is in contact with the composition for at least about 45 minutes. In some embodiments, the surface is in contact with the composition for at least about 30 minutes. In some embodiments, the surface is in contact with the composition for at least about 20 minutes. In some embodiments, the surface is in contact with the composition for at least about 10 minutes. In some embodiments, the surface is in contact with the composition for at least about 5 minutes.

In some embodiments, the surface to be sanitized is in contact with the composition for less than about 5 minutes. For example, the surface can be in contact with the composition for about 2 seconds, about 5 seconds, about 10 seconds, about 15 seconds, about 20 seconds, about 30 seconds, about 60 seconds, about 90 seconds, about 120 seconds, for about 3 minutes, or for about 4 minutes.

In some embodiments, the method further comprises adding a sufficient amount of aqueous solution or water to decrease the temperature and/or raise the pH of the composition. In some embodiments, the temperature of the composition is decreased to about 30° C. to 37° C. In some embodiments, the pH of the composition rises to a range of 4.5 to 7.0. In some embodiments, the addition of aqueous solution or water decreases the sanitizing activity of the composition. In some embodiments, the addition of aqueous solution or water reduces the enzymatic activity of an enzyme in the composition.

In some embodiments, the method further comprises rinsing the surface with an aqueous solution or water, wherein the rinsing results in a decrease in temperature and a rise in pH conditions. In some embodiments, the rinse step results in a decrease in temperature to about 30° C. to 37° C. In some embodiments, the rinse step results in a rise in pH to a range of 4.5 to 7.0.

In some embodiments, the rinse step results in a decrease in the sanitizing activity of the composition. For example, the rinse step can result in a decrease by about 25%, 30%, 35%, 40%, 45%, 50%, 55%, 60%, 65%, 70%, 75%, 80%, 85%, 90%, 95% or 100% of sanitizing activity relative to baseline (e.g., activity prior to the rinse step). In some embodiments, the rinse step can result in a decrease of at least about 25%, 30%, 35%, 40%, 45%, 50%, 55%, 60%, 65%, 70%, 75%, 80%, 85%, 90%, 95%, 98% or 99% of sanitizing activity relative to baseline. In some embodiments, the rinse step can result in a 100% decrease of sanitizing activity relative to baseline. In some embodiments, the rinse step can result in complete elimination of sanitizing activity relative to baseline.

In some embodiments, the rinse step results in a reduction in enzymatic activity of the composition. For example, the rinse step can result in a decrease by about 25%, 30%, 35%, 40%, 45%, 50%, 55%, 60%, 65%, 70%, 75%, 80%, 85%, 90%, 95% or 100% of enzymatic activity relative to baseline (e.g., enzymatic activity prior to the rinse step). In some embodiments, the rinse step can result in a decrease of at least about 25%, 30%, 35%, 40%, 45%, 50%, 55%, 60%, 65%, 70%, 75%, 80%, 85%, 90%, 95%, 98% or 99% of enzymatic activity relative to baseline. In some embodiments, the rinse step can result in a 100% decrease of enzymatic activity relative to baseline. In some embodiments, the rinse step can result in complete elimination of enzymatic activity relative to baseline.

In some embodiments, the rinse step results in a reduction in enzymatic activity of any enzyme remaining on the surface being sanitized. For example, the rinse step can result in a decrease by about 25%, 30%, 35%, 40%, 45%, 50%, 55%, 60%, 65%, 70%, 75%, 80%, 85%, 90%, 95% or 100% of enzymatic activity relative to baseline (e.g., enzymatic activity prior to the rinse step). In some embodiments, the rinse step can result in a decrease of at least about 25%, 30%, 35%, 40%, 45%, 50%, 55%, 60%, 65%, 70%, 75%, 80%, 85%, 90%, 95%, 98% or 99% of enzymatic activity relative to baseline. In some embodiments, the rinse step can result in a 100% decrease of enzymatic activity relative to baseline. In some embodiments, the rinse step can result in complete elimination of enzymatic activity relative to baseline.

In some embodiments, the composition is applied to soiled industrial equipment to clean and sanitize the industrial equipment. For example, the composition can be applied to a High Temperature/Short Time (HTST) units. HTST units are used in the dairy industry for processes that involve heating milk (or a dairy product) to a temperature of at least about 161° F. (71.7° C.) for at least about 15 seconds. Accordingly, in some embodiments, the composition can be applied to the surface of an HTST unit or vessel that is soiled with proteins, fats and mineral deposits, wherein contact between the composition and the surface of the HTST unit or vessel results in cleaning and/or sanitizing of the unit or vessel.

In some embodiments, the method of sanitizing comprises mechanical dish-washing. In some embodiments, the surface is placed in a dish-washing apparatus (e.g., dishwasher) prior to application of the composition to the surface. The dishwasher can be used to clean and sanitize cooking and eating articles, such as, e.g., dishes, bowls, cups, glasses, pots, pans, utensils and other cooking or food-serving equipment.

In some embodiments, the method of cleaning comprises a sanitize-in-place (SIP) or a sanitize-out-of-place (SOP) method. SIP systems include the internal components of industrial equipment such as tanks, lines, pumps and other equipment used for processing typically liquid product streams such as beverages, milk, and juices. SOP systems include readily accessible vessels of industrial equipment, including wash tanks, soaking vessels, holding tanks, scrub sinks, vehicle parts washers, noncontinuous batch washers and systems, and the like.

Automated sanitize-in-place (SIP) techniques have reduced the need for industrial equipment disassembly and increased the efficiency of cleaning and sanitizing methods. SIP techniques use the combination of chemistry and mechanical action to clean and sanitize the inside of industrial equipment without requiring the time consuming and labor intensive disassembly and manual cleaning of a system. SIP techniques generally include the circulation of chemistries (e.g., sanitizers, disinfectants, and the like) for periodic cleaning and sanitizing of industrial equipment. In some embodiments, SIP techniques involve a first rinse, the application of sanitizing solutions, a second rinse with potable water, followed by resumed operations. In some embodiments, one or both rinses are omitted. The process can also include any other contacting step in which a rinse, acidic or basic functional fluid, solvent or other cleaning component such as hot water, cold water, etc. can be contacted with the equipment at any step during the process.

Industrial equipment (e.g., brewery equipment or dairy equipment) can be sanitized or disinfected using SIP techniques. The sanitizing or disinfecting of the in-place systems can be accomplished with the compositions disclosed herein, and according to the present methods. In some embodiments, the compositions are heated prior to introduction into the in-place systems. For example, the compositions can be heated to about 50° C., 55° C., 60° C., 65° C., 70° C., 75° C., 80° C., 85° C., 90° C., 95° C., 100° C., 105° C., 110° C., or any temperature included between any two of these values. In some embodiments, a concentrated composition can be introduced into an in-place system (e.g., industrial equipment) and diluted, in situ, with water. In some embodiments, the composition is applied or introduced into the system as a use-solution. SIP techniques typically employ flow rates of about 0.1 meters per second to about 0.5 meters per second, about 1.0 meter per second, about 1.1 meters per second, about 1.2 meters per second, about 1.3 meters per second, about 1.4 meters per second, about 1.5 meters per second, about 1.6 meters per second, about 1.7 meters per second, about 1.8 meters per second, about 1.9 meters per second, about 2.0 meters per second, about 2.5 meters per second, about 3.0 meters per second, about 3.5 meters per second, about 4.0 meters per second, about 4.5 meters per second, about 5.0 meters per second or a range between and including any two of these values.

SIP techniques can employ the compositions disclosed herein with heated (e.g., about 50° C., 60° C., 70° C., 80° C., 85° C., 90° C., 95° C., 100° C., 105° C., 110° C.) water. SIP techniques can employ contact times of at least about 2 seconds, about 5 seconds, about 10 seconds, about 15 seconds, about 20 seconds, about 30 seconds, about 60 seconds, about 90 seconds, about 120 seconds, 5 minutes, 15 minutes, 30 minutes, one hour, two hours, or a range between and including any two of these values.

In some embodiments, the method of sanitizing further comprises collecting the composition as an effluent composition subsequent to contacting the surface being sanitized with the composition. Once the surface has been sanitized, and any soil or residue has dissolved or become suspended in the composition, the effluent composition is recovered. In some embodiments, the effluent composition is optionally concentrated by removal of liquid from the composition. In some embodiments, the effluent composition is filtered to remove any soil or residue that is not in solution.

In some embodiments, the effluent composition, whether directly recovered after sanitizing the surface or optionally concentrated after recovery, is stored in a holding vessel or container for from about 30 minutes to 8 days. For example, the effluent composition can be stored for at least 30 minutes, 1 hour, 2 hours, 4 hours, 6 hours, 8 hours, 12 hours, 18 hours, 24 hours, or for any duration of time between any two of these values. In some embodiments, the effluent composition can be stored for at least 1 day, 2 days, 3 days, 4 days, 5 days, 6 days, 7 days, or 8 days.

In some embodiments, the method of sanitizing further comprises recycling or reusing the effluent composition, wherein the effluent composition is contacted with a second surface to be sanitized. In some embodiments, the effluent composition is contacted with a second surface to be sanitized after being stored in a holding vessel or container for from about 30 minutes to 15 days. For example, the effluent composition can be contacted with a second surface to be sanitized after being stored for at least 30 minutes, 1 hour, 2 hours, 4 hours, 6 hours, 8 hours, 12 hours, 18 hours, 24 hours, or for any duration of time between any two of these values. In some embodiments, the effluent composition can be contacted with a second surface to be sanitized after being stored for at least 1 day, 2 days, 3 days, 4 days, 5 days, 6 days, 7 days, 8 days, 9 days, 10 days, 11 days, 12 days, 13 days, 14 days, or 15 days. In some embodiments, the effluent composition is contacted with a second surface to be sanitized after being recovered from the cleaning of a first soiled surface. In some embodiments, the effluent composition is contacted with a second surface to be sanitized within about 5 minutes, 10 minutes, 15 minutes, 20 minutes, 25 minutes or 30 minutes after being recovered from the disinfecting or sanitizing of a first surface. In some embodiments, the effluent composition is contacted with a second surface to be sanitized within about 5 minutes or directly after being recovered from the disinfecting or sanitizing of a first surface.

In some embodiments, the effluent composition can be reused at least once, twice, three times, four times, five times, six times, seven times, eight times, nine times, or ten times on separate surfaces to be sanitized. For example, the effluent composition can be reused at least once on a second surface, or reused a second time on a third surface, or reused a third time on a fourth surface, and so on.

In some embodiments, at least one enzyme is added to the effluent composition prior to its contact or reuse for cleaning on the separate surfaces to be sanitized. For example, at least one enzyme can be added to the effluent composition prior to its reuse and contact with a second surface, or prior to its reuse and contact with a third surface, or prior to its reuse and contact with a fourth surface, and so on. In some embodiments, the at least one enzyme can be added to the effluent composition prior to each time it is reused for application on a surface to be sanitized. In some embodiments, the at least one enzyme can be added to the effluent composition prior to every other time it is reused for application on a surface to be sanitized. The at least one enzyme can be added in any amount as disclosed herein, for example, from about 0.0001 mg to 1000 mg of enzyme protein per 100 grams of soil on the surface, or from about 0.0001 wt % to 50 wt % of the effluent composition, or provided in an activity range of from about 0.0001 to 100 activity units.

### Kits

Also provided herein are kits for cleaning or sanitizing a surface, wherein the kit comprises: an enzyme or enzyme mixture, an acid, optionally one or more additives, and instructions for their use. The enzyme or enzyme mixture can be a thermophilic, hyperthermophilic and/or acidophilic enzyme as described herein. The acid and optional additive can be any acid and additive as disclosed herein.

In some embodiments, the enzyme or enzyme mixture is provided as a lyophilized product, which can optionally be provided with a diluent. In some embodiments, the enzyme or enzyme mixture is provided as a suspension. In some embodiments, the enzyme or enzyme mixture is provided as a solution. In some embodiments, the enzyme or enzyme mixture is provided in one container, and the optionally provided diluent is provided in a second, separate container. In some embodiments, instructions for preparing the enzyme or enzyme mixture in the optionally provided diluent are provided.

In some embodiments, the enzyme or enzyme mixture, the acid and the optional additive(s) are provided in separate, individual containers. In some embodiments, the enzyme (or enzyme mixture) and the acid are provided in the same container, and the optional additive(s) are provided in a separate container. In some embodiments, the acid and optional additive(s) are provided in the same container, and the enzyme (or enzyme mixture) is provided in a separate container.

In some embodiments, the kits can be stored at ambient (about 20° C.-25° C.) temperatures. In some embodiments, the kits can be stored at about 4° C. In some embodiments, the kits can be stored at temperatures of from about 4° C. to about 20° C. In some embodiments, the kits can be stored at temperatures of up to about 30° C.

In some embodiments, the kits have a storage shelf-life of at least about three months. In some embodiments, the kits have a storage shelf-life of at least about six months. In some embodiments, the kits have a storage shelf-life of at least about nine months. In some embodiments, the kits have a storage shelf-life of at least about 12 months, 18 months, 24 months, 30 months or 3 years.

### EXAMPLES

### Example 1

### Production of Candidate Thermophilic Enzymes

Potentially useful gene sequences were identified using standard bio-informatics approaches. Genes of interest were isolated and cloned using standard molecular biology techniques according to a scheme similar to those disclosed in WO 2014/018973, which is incorporated herein by reference in its entirety. Functional enzymes were produced by recombinant expression in hyperthermophilic and acidophilic microbes of the domain Archaea of the order Sulfolobales. Transformed microbes were cultured at 80° C. and pH =3.0, and culture medium included carbon, nitrogen, phosphorous, and sulfur sources and trace minerals. Genetic constructs of genes of interest were designed to target gene products to the extracellular space using localization sequences similar to those described previously (WO 2014/018973). Recombinant enzymes accumulated in the culture media and were concentrated and buffer exchanged using commercially available tangential flow filtration devices. In some embodiments, enzymes were designed to have an epitope, a poly-histidine fusion (e.g., a histidine tag) or another useful modification to facilitate purification and/or characterization. Enzymes were concentrated 200-10,000× from the original solution and filter sterilized and stored at room temperature, −20° C., −80° C. or lyophilized.

Enzymes suitable for acidic pH environments have at least 25% of their maximum activity at pH values ranging from about 0.5 to 4.5. Exemplary optimum activities range from about pH 2.5 to 3.5. Enzymes suitable in neutral pH environments have at least 25% of their maximum activity pH values ranging from about 4 to 7. Exemplary optimum activity for such an enzyme can be at about pH 5.5.

Enzymes suitable for hyperthermophilic environments have at least 25% of their maximum activity at temperatures ranging from about 70° C. to about 110° C. Exemplary optimum activities range can be from about 70° C. to about 90° C., or from about 75° C. to about 85° C., or at about 80° C.

### Example 2

### Characterization Of Candidate Enzymes

Enzymes of interest (Example 1) were profiled for operational activity across a range of pH values (0-7) and temperatures (50° C.-110° C.) using standard biochemical methods and assays appropriate for the specificity of each enzyme. Enzymes of interest were next tested on a series of substrates from a variety of sources. In the case of protease enzymes, substrates included, for example, hemoglobin, casein, whole milk, yogurt, whey protein, bovine serum albumin, and the like. Substrate testing was used to assess the range of substrate specificity and to accurately assess the applicability of each enzyme for cleaning efficacy of various industrial soils. In the case of the glycohydrolase enzymes, xylans, cellulose, crystalline cellulose, corn stover, switchgrass, juice pulps, and other plant materials were tested.

Enzymes of interest were also characterized for the impact of a series of commonly used detergents, surfactants, and in various acids phosphoric, nitric, acetic, sulfuric, hydrochloric, citric, paracetic and mixtures thereof on enzymatic activity at their respective optimal temperature and pH. These studies were used to identify formulation components compatible with each individual enzyme and its optimal activity.

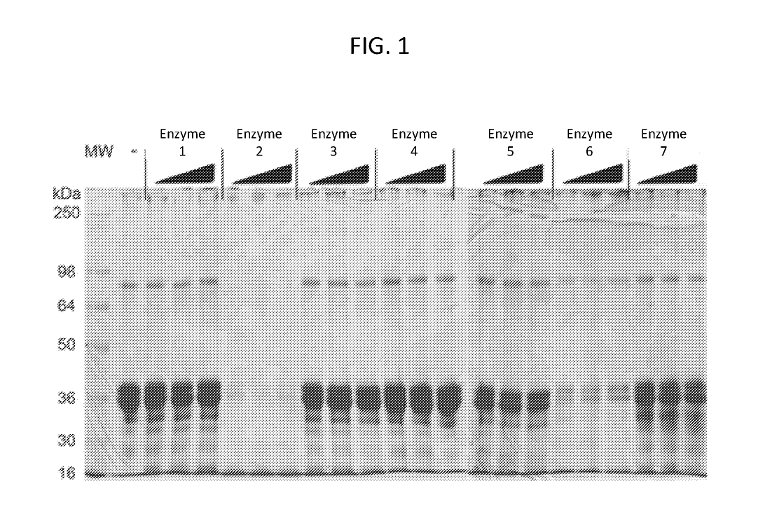

As an example, seven recombinant extremophilic protease enzyme candidates were screened for activity on dairy industry-specific substrates (e.g., milk proteins). As comparison benchmarks, commercial enzyme formulations currently used for membrane defouling were also tested. Preliminary data indicated two promising candidate enzymes (Enzyme 2, Enzyme 6; FIG. 1), which were subsequently scaled up and produced in quantities suitable for field tests at a commercial creamery as described below.

In addition to determining the enzymes suitable for use in dairy applications, optimal pH and thermal ranges were characterized to establish operational parameters available for industrial cleaning and sanitation protocols. Enzyme activity was also tested in various acids and detergents, and the half-life of the enzymes of interest was determined to further define the variables that could be exploited to achieve the most effective cleaning protocols.

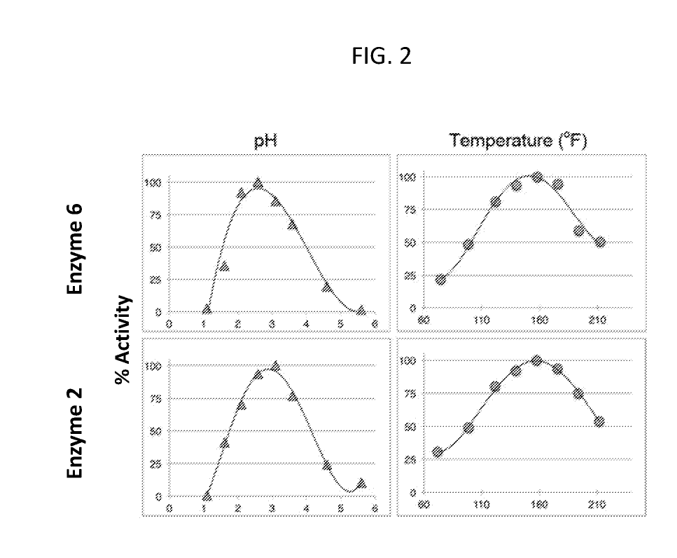

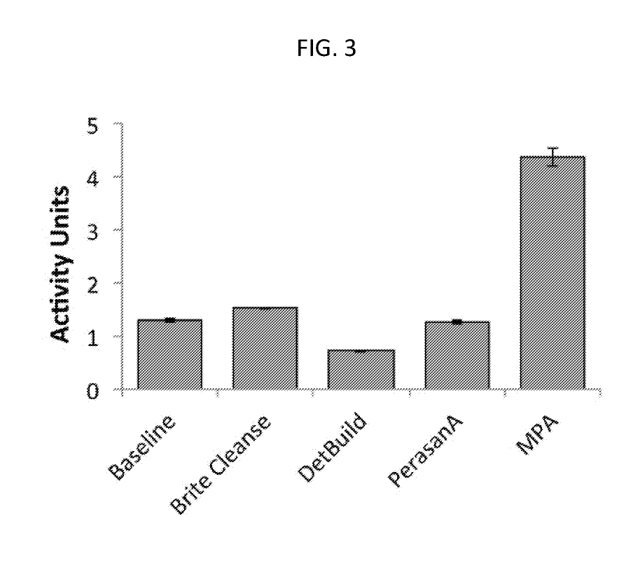

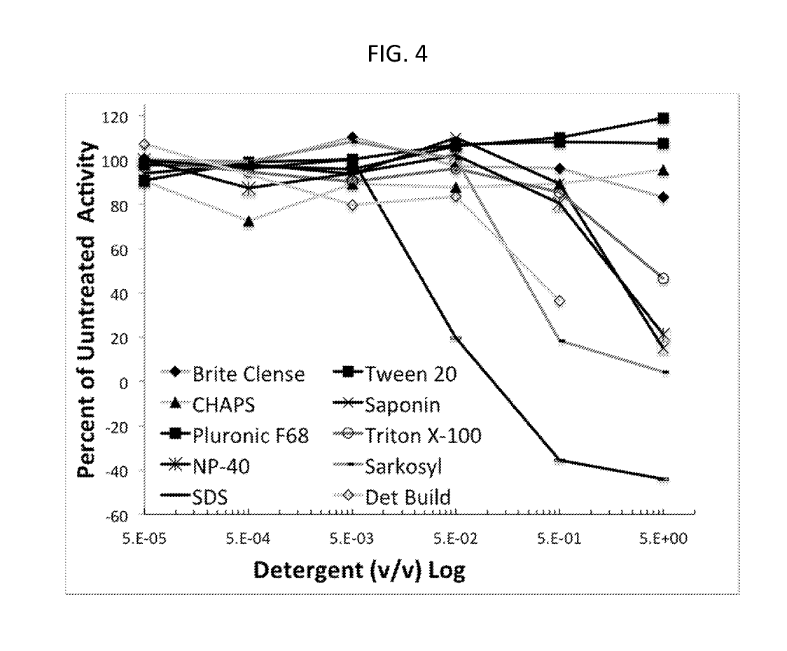

As an example, quantitative protease enzyme assays were run across a pH range of from about 1 to 5.8 and at temperatures ranging from about 75° F.-220° F. (about 24° C.-105° C.). The percent maximal activity was plotted to identify functional ranges and optimal conditions for activity (FIG. 2). Various additives (Hydrite Chemical Co.) were also tested for effects on enzyme activity to determine which chemicals are compatible with both the candidate enzymes and an industrial cleaning process used by a dairy client. MPA No. 168 (45% nitric acid, 5% phosphoric acid, percentages by weight) was identified as an acid used in the dairy client's cleaning process that was compatible with high candidate enzyme activity (FIG. 3, representative data). Enzyme activities were also tested in increasing concentrations of a set of ten surfactants or detergents (FIG. 4, representative data). In addition, the half-lives for top candidate protease enzymes was determined to be 6 or more days at pH=3 and 175° F. (79.4° C.), which is remarkably long for an enzyme under such conditions. Notably, the half-lives for the top two candidate protease enzymes was determined to be 10 and 15.5 days at pH=3 and 175° F. (79.4° C.). From the sum of these data sets, an enzyme formulation was identified and further developed with acids that are compatible with creamery operations, and the thermal and pH ranges practical for cleaning protocol development with the formulation was determined. A commercial acid mixture (MPA No. 168: 45% nitric acid, 5% phosphoric acid, percentages by weight) that is currently in use for dairy cleaning at pH 3-4 and a temperature of 175° F. was selected for formulation with a mix of two candidate enzymes that showed peak activity on milk proteins in these conditions. These formulations and conditions were tested and validated at laboratory scales (0.5-1 ml) to conserve enzymes and allow rapid screening and fine tune the formulation and pilot protocol conditions.

### Example 3

### Composition Formulation

Because a large portion of industrial equipment is made from 316 stainless steel or a lesser grade of stainless steel, many formulations are initiated with nitric acids or common mixtures of nitric and phosphoric acids, which are less damaging to the stainless steel equipment. Enzymes compatible with these acids were selected and tested with laboratory scale tests designed to closely mimic the industrial application of interest. Formulations of acids, detergents and enzymes were assayed for activity at temperatures ranging from 50° C.-105° C., and at pH 1.5-4.5 in laboratory assays for each given application.

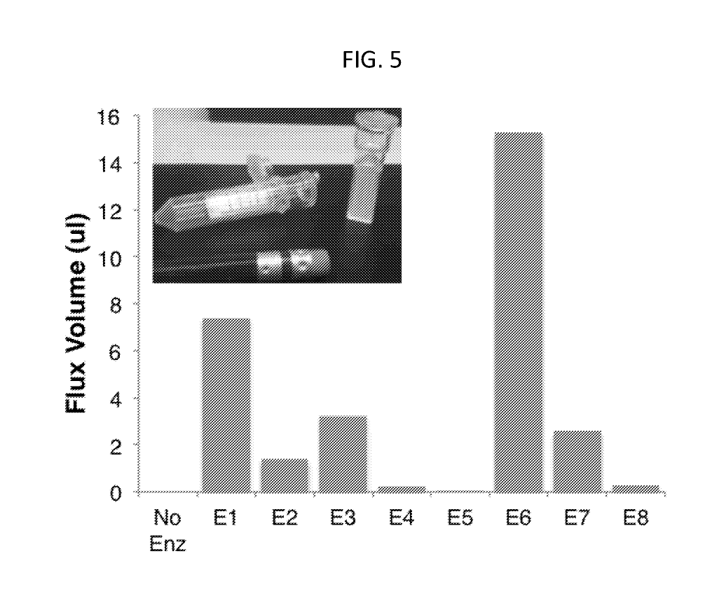

In one exemplary embodiment, a membrane defouling formulation was developed. Polyethersulfone (PES) membranes are the industry standard used for most industrial ultrafiltration systems. In the dairy industry settings, the membrane fouling decreases the flow of liquid through the PES membrane, resulting in reduced flux. PES is also used in commercial small-scale laboratory disposable centrifugal filters (FIG. 5, inset). Accordingly, these small-scale filters were ‘fouled’ with dairy products (e.g., milk, yogurt, whey protein), treated with various enzyme, detergent and acid formulations, and efficacy of the treatment was evaluated by recording the recovery of flux. These pilot studies were used to rapidly establish approximate enzyme dosage, treatment times and temperatures, specific enzymes, and screen detergent (surfactant) efficiencies in removing milk protein fouling of PES membranes. One such data set is shown in FIG. 5, where a series of 8 different candidate enzymes were screened for membrane defouling at pH=3.5 in a mix of nitric and phosphoric acids at 80° C. for one hour. These data show a varied performance in defouling achieved by enzymes 1-8 with E1 and E6 showing the greatest recovery in flux (i.e., the most amount of membrane defouling). These initial screens were followed by additional testing in industry standard equipment for evaluating membrane defouling activities. Finally these formulations were field tested for defouling membranes used at a production facility with standard production process and products.

### Example 4

### Field-Testing of Candidate Formulations: Membrane Defouling

Candidate protease enzymes identified as highly active on milk proteins (Examples 2, 3) were field-tested at a dairy client for efficacy in cleaning and sanitizing dairy equipment. Trials were set up using an apparatus that operates with 12-15 gallons of liquid, a 1/10 scale mimic of the production equipment used in the creamery for a set of specific products. This equipment is routinely used at the creamery to vet all their clean-in-place (CIP) protocols and is their last test prior to full production line implementation. The membrane used has a surface area of 7.3 square meters (79 square feet) and is designed specifically to tolerate high temperatures and pressures (KOCH membrane, model #HpHT 4336-K131-VYV). Several different readouts for cleaning efficiency were measured during the trials to assess the cleaning efficacy. To assure a realistic measure of the enzymatic cleaning processes, the following measurements were also quantified: the total protein in solution, the amount of degraded protein, the amount of protein removed from the membrane, and the membrane flux during and after cleaning.

To make a fair comparison between current industrial protocols and the proposed cleaning protocols with candidate enzyme formulations, the membrane manufacturer's cleaning protocol was employed and used with the candidate enzyme formulations, which included chemicals available from Hydrite Chemical Co., the current service contractor for the dairy's creamery CIP processes. Exemplary chemicals used in the manufacturer's protocol include Bright Cleanse No. 321 (Hydrite Chemical Co., Product No. FP032101), MPA No. 168 (Hydrite Chemical Co., Product No. FP016801), Hydrizyme No. 399 (Hydrite Chemical Co., Product No. FP039902), and Hydriflux NP (Hydrite Chemical Co., Product No. FP036601).

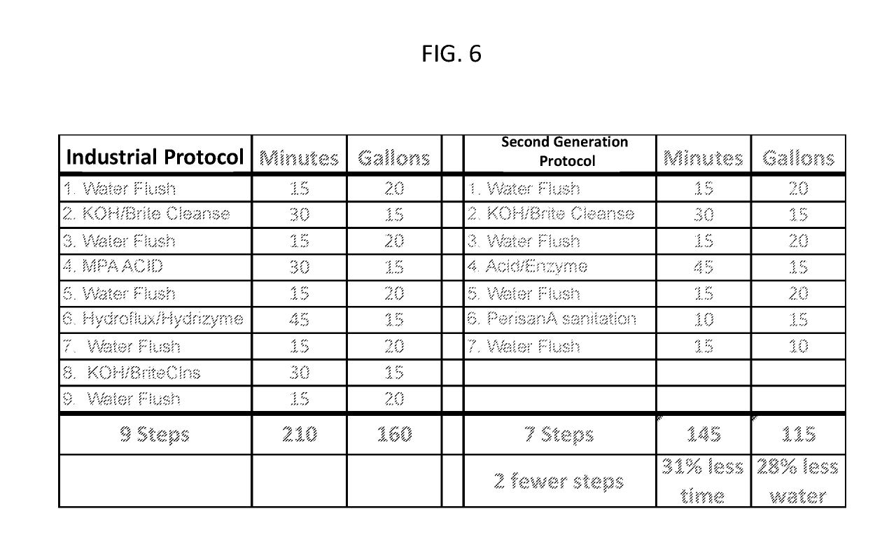

For the first generation proposed cleaning protocol, the current CIP process was adapted to take advantage of the unique capabilities of the candidate enzyme formulations. The adapted manufacturer's protocol became the first generation proposed dairy membrane cleaning protocol using the candidate enzyme formulations. After the initial trials, the first generation protocol was amended and optimized to develop the second generation proposed cleaning protocol using the enzyme formulations. The second generation protocol and its improvements over the current industrial protocols are summarized and compared in the table in FIG. 6.

The table in FIG. 6 indicates that the second generation protocol realized a 28% savings in water and 31% savings in time while delivering an equal or better membrane flux recovery (cleaning efficiency) relative to the manufacturer's CIP protocol currently in use at the dairy. This represents a significant reduction in water consumption and cleaning time.

To more fully characterize the performance of candidate enzymes in defouling applications, samples were manually collected during the treatment protocol steps from the recirculating CIP vessel. Because the fouling and caustic steps (Steps 1 to 3) were identical between the manufacturer's protocol and the proposed second generation protocol (See Table 1), the focus was on determining the contribution of the steps unique to the second generation enzymatic formulation protocol.

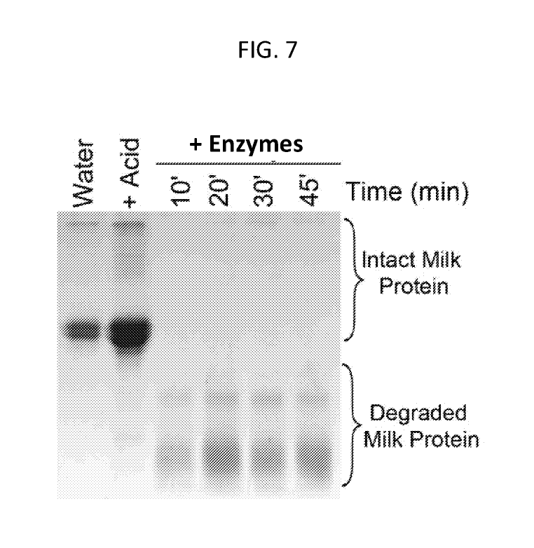

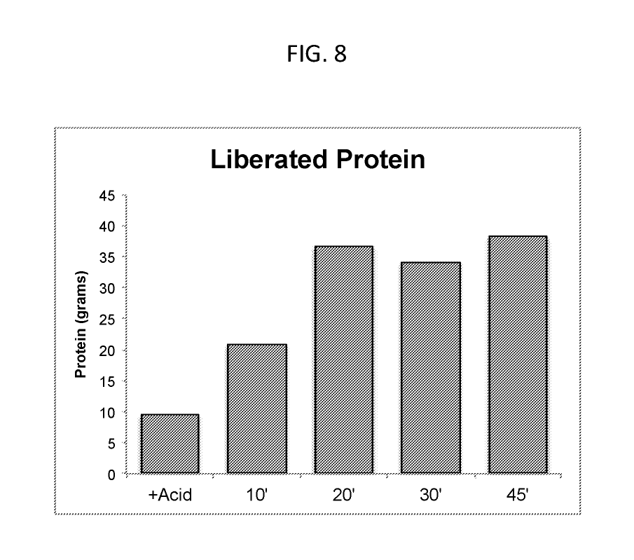

Milk proteins from the trials were first evaluated using standard SDS-PAGE gels stained with coomassie brilliant blue and visualized from time points across the course of the trial (FIG. 7). The results indicate that: 1) the addition of acid in the MPA product loosens fouling proteins and increases the amount of milk proteins in the circulation vessel (lanes 1, 2), and 2) addition of the enzymes degrade the vast majority of proteins in the system within the first 10 minutes of treatment (lanes 3-6). To verify the results from the SDS-PAGE gels, the total protein in solution was quantified using the Lowry protein assay for the same time points (FIG. 8). Because the protein of interest (the protein fouling the membrane) is not in the recirculating mix, any increase in the total protein in solution in this closed system is due to protein that is being removed from the equipment and liberated into solution. Accordingly, based on the measured total protein amounts in the circulating, it was determined that the acid and the enzymes were having the desired effect. Specifically, upon addition of acid, 9.5 grams of protein was liberated, with an additional 11 grams being liberated in the first 10 minutes of enzyme treatment, and an additional 15.8 grams liberated after 20 minutes of enzyme treatment (26.8 grams of total protein removed by enzymes). Taken together, these analyses demonstrate that treatment of the approximately 80 square foot fouled PES membrane liberated over 35 grams of protein in approximately 20 minutes and restored flux to baseline levels. Notably, the trial enzyme treatment was carried out for 45 minutes; however, the analyses by SDS-PAGE gel and Lowry assay reveal that a 20-minute treatment with acid and candidate enzymes can be sufficient to defoul membranes and restore flux at the administered amounts. This reduction in treatment time could save an additional 25 minutes and increase the membrane cleaning time savings to 43% from 31%. Further reductions in treatment times and/or improvements in cleaning efficacy are possible with adjustment of operational parameters.

### Example 5

### Biofilm Sanitation Methods

Biofilms are one of the most challenging forms of microbial contamination sanitize or remove from equipment of many types and the source of much concern in the food processing industry. The difficulty in removing biofilms is, in part, due to the large amount of extracellular material (primarily sugar polymers and proteins) that form a barrier to protect cells from chemical, mechanical, and enzymatic interventions. Enzymes can be formulated to specifically attack this protective layer of biomolecules and expose the cells inside to killing agents of all kinds. In this example, a mixture of enzymes was developed for sanitation protocols at high temperatures (70° C.-120° C.) and in the presence of harsh acids (e.g., sulfuric acid) to degrade the protective layer on biofilms and simultaneously expose the biofilm cells to acid, heat, enzymes and sanitizing chemicals.

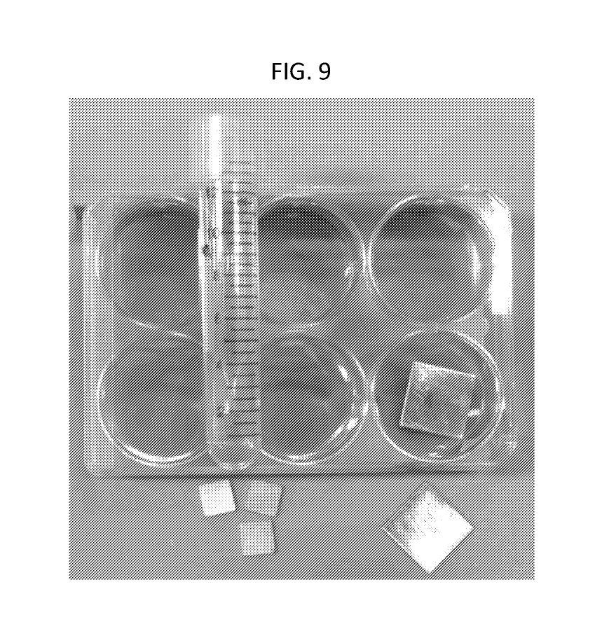

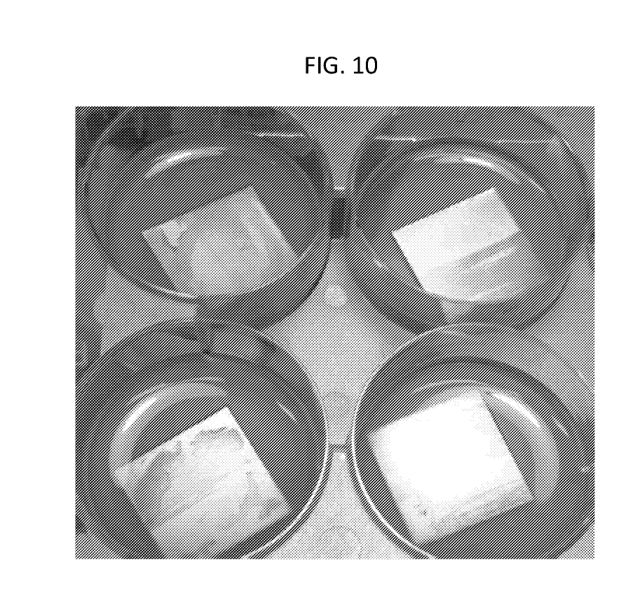

Quantifying biofilm sanitation. Biofilms were grown on stainless steel coupons and then treated with various agents and remaining cells dislodged and plates on petri dishes to get a viable cell count (Donelli, G (editor). 2014. Microbial biofilms: Methods and Protocols, 1^{st} edition. Springer:New York; Burgess, et al. 2014. “Biofilms of thermophilic bacilli isolated from dairy processing plants and efficacy of sanitizers.” Methods Mol Biol 1147:367-377). Laser-cut standard coupons made of 316-grade stainless steel were obtained to establish assays for biofilm removal and sanitation (FIG. 9). Cultures of various bacterial species were grown to determine conditions that promote biofilm formation, and resulting biofilms were visually observed on the larger stainless steel coupons (FIG. 10). Microbial strains tested included Enterococcus faecium, Streptococcus mutans, Clostridium species, Listeria monocytogenes and E. coli O157:H7. Of particular note, both Listeria monocytogenes and E. coli O157:H7 form biofilms on food processing equipment in dairies and meat processing and are known sources of foodborne illness.

Using standard biofilm quantitation methods with a modified outgrowth protocol, the efficacy of enzyme treatments in hot acidic conditions were quantified. In one particular embodiment, a protease:glycohydrolase enzyme mixture was applied. The mixture of acid- and heat-stable glycohydrolase and protease enzymes in sulfuric acid (pH of composition=3.0) was applied to biofilm-colonized 316-grade stainless steel coupons at a temperature of 80° C. for 5 minutes. After the treatment, the coupons were thermally quenched on an ice bath. Coupons were bead-beaten for 1 minute in rich microbial media to dislodge cells, and serial dilutions of the resulting liquid was plated in triplicate and incubated overnight at 37° C. After the overnight incubation, resulting colonies were scored.

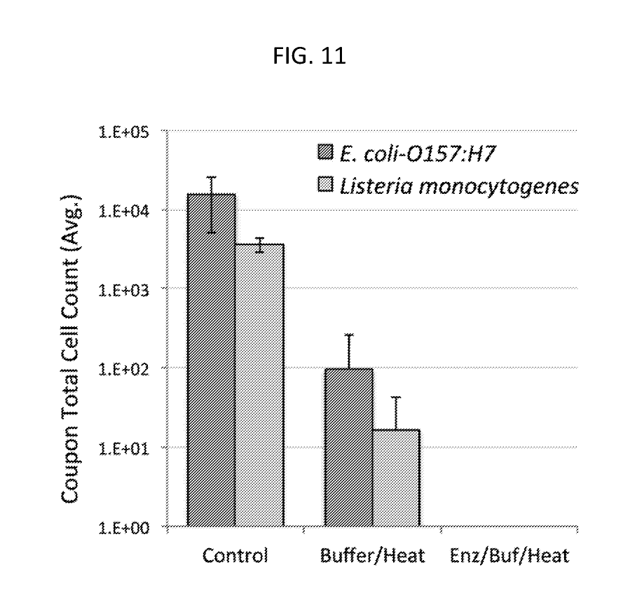

The results of the sanitation treatment indicated very effective microbial sanitation of the stainless steel coupons (FIG. 11). Remarkably, not even a single viable microbial cell was observed after treatment of the colonized coupons with the candidate enzyme mixture, indicating that the enzyme addition markedly improved sanitation of biofilms.

In conclusion, it was demonstrated that a combination of enzymes, acids and extreme heat are an effective means for biofilm sanitation.

### Example 6

### Enzyme Shelf Life and Storage Studies

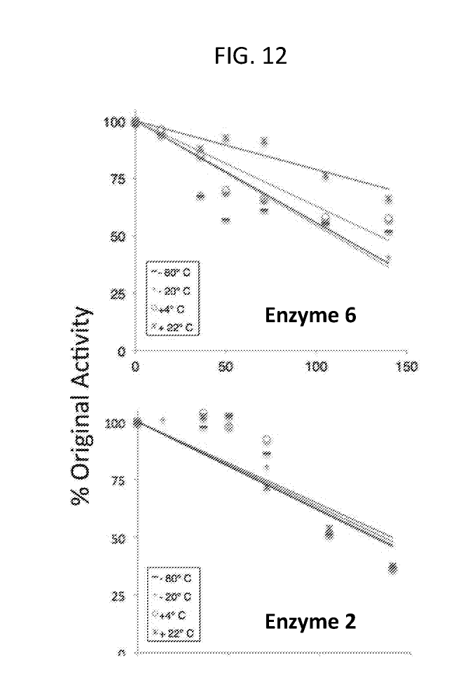

A series of shelf-life studies were carried out on candidate enzymes. In a first experimental set, the activity of two protease enzymes was measured over a 140-day trial under various typical enzyme storage conditions. The conditions tested were −80° C., −20° C., 4° C., and ambient temperature (22° C.-25° C.) either with or without cryprotectant (10% glycerol). Enzymes were portioned into small aliquots and stored at the various temperatures and assayed for activity at various time points across the 140-day trial. The activity at the time-points was calculated as a percent of the starting activity and plotted versus time (FIG. 12). Both the individual data points and the linear regression of the series show a difference between the two different enzymes with respect to storage stability. Enzyme 6 in this study showed a more clear dependence on storage temperatures, with ambient temperatures retaining over 65% of activity at the 140 day time point. Enzyme 2 showed a different profile with nearly identical results at all temperature conditions tested. Surprisingly, the addition of glycerol as a cryprotectant diminished shelf life to 30-40% of the original activity by the 140 day time point at all temperatures, and has therefore been discontinued as a common practice (data not shown). Given that enzyme mixtures will eventually be formulated for market, and the fact that individual enzymes respond differently to most storage conditions, further investigations were carried out to optimize general enzyme storage regimens.

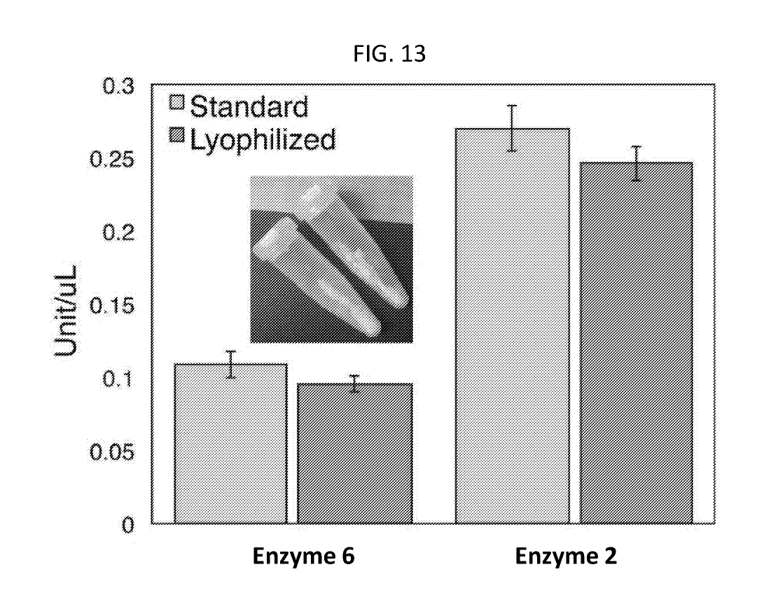

Dessication of enzymes using lyophilization (freeze drying) is an effective long-term storage processes for enzymes and other proteins as well as food products. To test whether the candidate enzymes are amenable to this storage method, two of the candidate protease enzymes used for the dairy trials (Example 3, 4) were freeze-dried, and aliquots of the lyophilized enzymes were reconstituted after storage for one week at ambient temperature to determine the retention of activity after lyophilization treatment. It was determined that almost 90% of the enzymatic activity is retained relative to standard conditions (i.e., storage in solution at ambient temperatures) in both candidate protease enzymes after lyophilization and reconstitution (FIG. 13). Notably, these two enzymes showed very different storage properties at all the temperatures tested in the first shelf-life study, but behave almost identically with respect to activity after freeze drying (FIGS. 12, 13). While long-term storage of enzymes in a freeze dried state is effective and can be useful for storage times on the scale of years, not all enzymes are amenable to freeze drying. Accordingly, the data from the lyophilization shelf-life studies indicates that the candidate enzymes can be suitable for commercial formulation packing, storage, and shipping. Longer term shelf-life studies of freeze-dried candidate enzymes are currently ongoing.

### Example 7

### Thermostable Lipase Enzymes

Lipase enzymes of interest (Example 1) were investigated for activity for operational activity at high temperature and neutral pH.

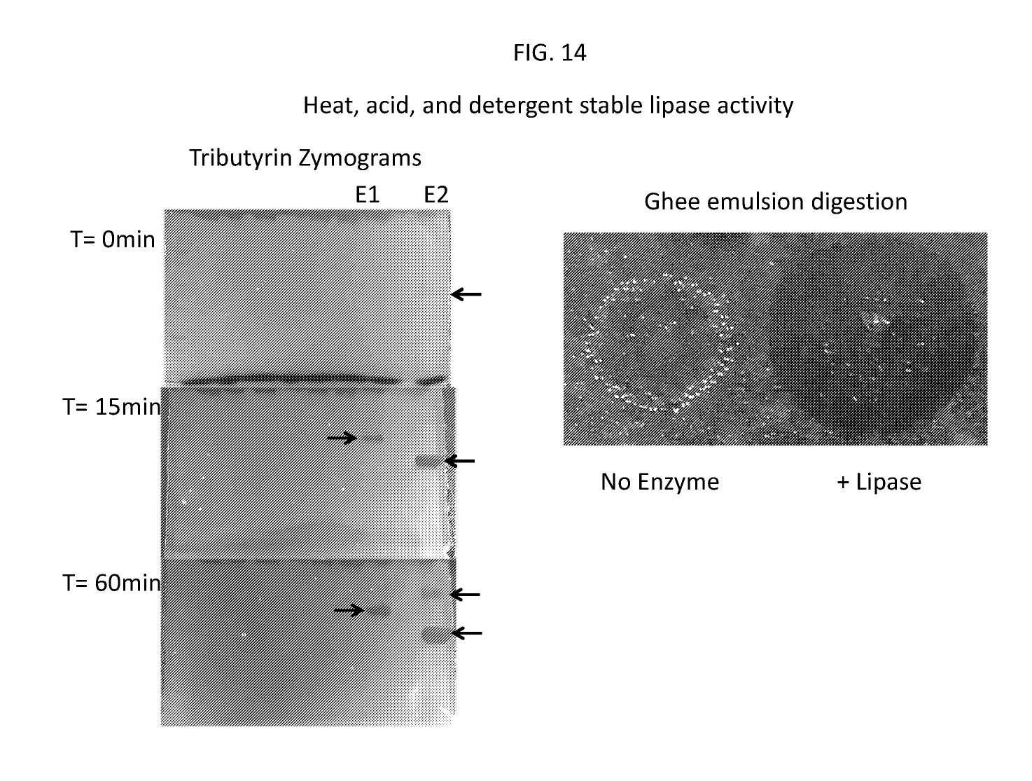

In one experiment, portions of two purified thermostable lipases (5 microliters) were electrophoresed on 10% SDS-PAGE gels containing 2% sodium dodecyl sulfate and embedded with an emulsion of the pure fatty acid tributyrin (FIG. 14, left). After electrophoresis, the gels were incubated at 80° C. in a buffer solution with a pH of 6.0 for the noted times (0, 15, and 60 minutes). Enzymatic activity is present when a clearing of the tributyrin emulsion occurs; the triacylglycerides are degraded and fat globules embedded in the gels are dissipated only in the location of active lipase enzymes. Arrows indicate the observed mobility of the active forms of the two enzymes (E1 and E2) and the associated clearing due to lipase activity.

In a second experiment, a thermostable lipase was applied to a petri dish filled with gellan gum (pH 6) entrapping an emulsification of milk fats (ghee, which contains mixed triacylglycerides). Two 10-microliter spots were applied (+/−enzyme) to the petri dish as indicated (FIG. 14, right), and the plate was incubated at 80° C. for 90 min. When visualized against a black background, the section of petri dish to which lipase enzyme was applied showed an apparent clearing of mixed triacylglycerides, indicating activity of the lipase enzyme at a temperature of 80° C.

These results demonstrate that lipase enzymes isolated from hyperthermophilic enzymes activity exhibited observable activity at elevated temperatures such as 80° C. Such enzymes illustrate useful potential in cleaning and sanitizing processes for dairy equipment at elevated temperatures.

### Example 8

### Thermostable Lipase Enzymes

Protease enzymes of interest (Example 1) were investigated for activity for operational activity at various temperature and pH values.

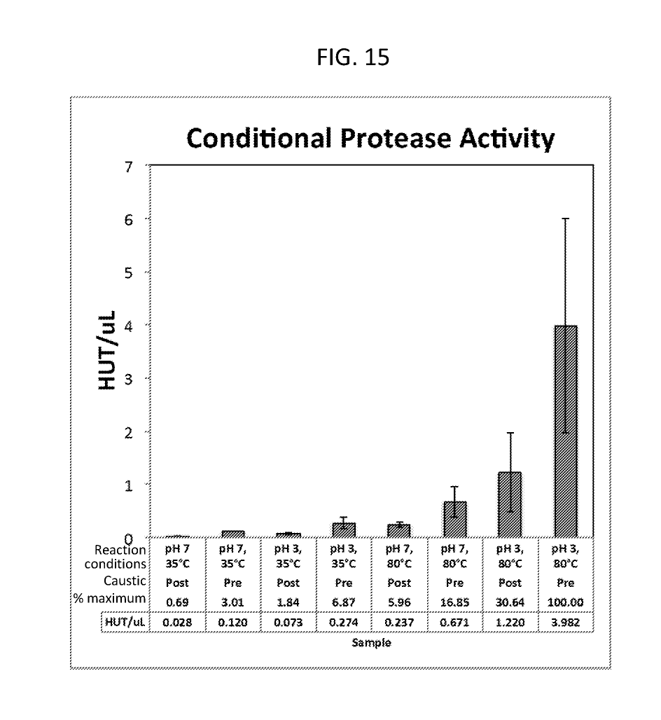

Hemoglobin substrate was digested in standardized liquid assays by a constant amount of two exemplary protease enzymes formulated for dairy cleaning applications. Units of activity (hemoglobin units tyrosine, HUTs) were quantified from triplicate reactions at the indicated series of conditions (pH 3 or 7, temperature of 35° C. or 80° C., FIG. 15). To assess the effect on enzymatic activity of a caustic wash as part of the cleaning process, a subset of reactions was adjusted to pH 10 with sodium hydroxide (NaOH), incubated at 80° C. for 60 minutes, and assayed for activity (“Post”). Control reactions were those reactions not exposed to pH 10 by addition of NaOH (“Pre”). The percentage of maximal activity under optimal conditions was calculated and is indicated for each tested condition at the bottom of the graph (“% maximum”).

These results (FIG. 15) demonstrate that protease enzymes isolated from hyperthermophilic and acidophilic sources illustrated highest activity at acidic pH (pH 3) and elevated temperatures (80° C.), with decreased activity observed as a result of (i) addition of sodium hydroxide to increase the reaction pH to 10, (ii) reaction conditions at pH 7, (iii) reaction conditions at lower temperature (35° C.), or (iv) a combination of (i), (ii), and (iii). This indicates that the enzymatic activity for hyperthermophilic and acidophilic protease enzymes, which is highest at acidic pH and temperatures of about 80° C., can be quenched by adjusting conditions for the reaction mixture to neutral or basic pH and lowering temperatures to less than about 50° C.

### Example 9

### Cleaning a Surface of Grain Residue

A composition containing an enzyme isolated from a hyperthermophilic and/or acidophilic organism, or a composition containing an enzyme mixture containing two or more enzymes isolated from a hyperthermophilic and/or acidophilic organism, is provided. In the presence of an acid (e.g., MPA No. 168 (Hydrite Chemical Co., Product No. FP016801, 45% nitric acid, 5% phosphoric acid, percentages by weight), the composition is placed in contact with a soiled surface containing grain residue. The composition is optionally heated to a temperature of at least about 70° C. prior to contact with the soiled surface. After placing the surface in contact with the composition for a period of time of at least about 5 minutes, the composition is collected, and the surface is rinsed with water and/or a solution with a basic pH (pH of about 9 or above). The water and/or basic pH solution for rinse are optionally cooled to a temperature of about 10° C. to 25° C. prior to contact with the surface. Visual inspection of the surface indicates that no grain residue remains on the surface.

The collected composition is stored in a holding vessel, and the composition is reused on a second soiled surface containing a grain residue in the presence of an acid. Optionally, additional enzyme is added to the composition prior to its reuse in cleaning the second soiled surface.

### Example 10

### Cleaning a Surface Used to Produce a Biofuel

A composition containing an enzyme isolated from a hyperthermophilic and/or acidophilic organism, or a composition containing an enzyme mixture containing two or more enzymes isolated from a hyperthermophilic and/or acidophilic organism, is provided. In the presence of an acid (e.g., nitric acid, phosphoric acid, sulfuric acid, or a combination thereof), the composition is added to a vessel used in the production of a biofuel as part of a clean-in-place (CIP) protocol. The composition is optionally heated to a temperature of at least about 70° C. prior to addition to the vessel. After contacting the surface of the vessel with the composition for a period of time of at least about 15 minutes, the composition is collected, and the vessel walls are rinsed with water and/or a solution with a basic pH (pH of about 9 or above). The water and/or basic pH solution for rinse are optionally cooled to a temperature of about 10° C. to 25° C. prior to contact with the vessel wall. Visual inspection of the vessel surface indicates that no biofuel production residue remains on the surface.

The collected composition is stored in a holding container, and the composition is reused in the presence of an acid on a second vessel to be cleaned. Optionally, additional enzyme is added to the composition prior to its reuse in cleaning the second vessel.

### Example 11

### Sanitizing a Surface Used to Brew Beer

A composition containing an enzyme isolated from a hyperthermophilic and/or acidophilic organism, or a composition containing an enzyme mixture containing two or more enzymes isolated from a hyperthermophilic and/or acidophilic organism, is provided. In the presence of an acid (e.g., nitric acid, phosphoric acid, sulfuric acid, or a combination thereof), the composition is added to a fermentation vessel used to brew beer as part of a sanitize-in-place (SIP) protocol. The composition is optionally heated to a temperature of at least about 70° C. prior to contact with the fermentation vessel. After placing the surface in contact with the composition for a period of time of at least about 20 minutes, the composition is collected, and the fermentation vessel walls are rinsed with sterile water and/or a sterile solution with a basic pH (pH of about 9 or above). The sterile water and/or sterile basic pH solution for rinse are optionally cooled to a temperature of about 10° C. to 25° C. prior to contact with the fermentation vessel. Swab testing of the vessel surface indicates that no living microorganisms remain on the surface.

The collected composition is stored in a holding tank, and the composition is reused in the presence of an acid on a second fermentation vessel to be sanitized. Optionally, additional enzyme is added to the composition prior to its reuse in sanitizing and/or disinfecting the second fermentation vessel.

### Example 10

### Sanitizing a Surface Used to Produce a Pharmaceutical Product

A composition containing an enzyme isolated from a hyperthermophilic and/or acidophilic organism, or a composition containing an enzyme mixture containing two or more enzymes isolated from a hyperthermophilic and/or acidophilic organism, is provided. In the presence of an acid (e.g., nitric acid, phosphoric acid, sulfuric acid, or a combination thereof), the composition is added to a tank used in the production of a pharmaceutical product as part of a sanitize-in-place (SIP) protocol. The composition is optionally heated to a temperature of at least about 70° C. prior to contact with the tank surface. After contacting the surface of the tank with the composition for a period of time of at least about 25 minutes, the composition is collected, and the tank walls are rinsed with sterile water and/or a sterile solution with a basic pH (pH of about 9 or above). The sterile water and/or sterile basic pH solution for rinse are optionally cooled to a temperature of about 10° C. to 25° C. prior to contact with the tank walls. Swab testing of the tank surface indicates that no living microorganisms remain on the surface of the tank walls.

The collected composition is stored in a holding container, and the composition is reused in the presence of an acid on a second tank or vessel to be cleaned. Optionally, additional enzyme is added to the composition prior to its reuse in sanitizing and/or disinfecting the second tank or vessel.

### Example 11

### Sanitizing a Surface to Inhibit Recolonization

A composition containing an enzyme isolated from a hyperthermophilic and/or acidophilic organism, or a composition containing an enzyme mixture containing two or more enzymes isolated from a hyperthermophilic and/or acidophilic organism, is provided. In the presence of an acid (e.g., nitric acid, phosphoric acid, sulfuric acid, or a combination thereof), the composition is added to surface to be sanitized. The composition is optionally heated to a temperature of at least about 70° C. prior to contact with the surface to be sanitized. After contacting the surface of the vessel with the composition for a period of time of at least about 30 minutes, the composition is collected, and the surface is rinsed with sterile water and/or a sterile solution with a basic pH (pH of about 9 or above). The sterile water and/or sterile basic pH solution for rinse are optionally cooled to a temperature of about 10° C. to 25° C. prior to contact with the surface. Swab testing of the surface indicates that no living microorganisms remain on the surface. Swab testing on the surface at seven days after the sanitizing procedure indicates that no additional microorganisms colonize on the sanitized surface.

One or more features from any embodiments described herein or in the figures may be combined with one or more features of any other embodiments described herein or in the figures without departing from the scope of the invention.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A composition for cleaning or sanitizing a surface, comprising an enzyme isolated from a hyperthermophilic organism and an acid.

2. The composition of claim 1, further comprising a surfactant or detergent.

3. The composition of claim 1, wherein the acid is selected from the group consisting of: nitric acid, phosphoric acid, hydrofluoric acid, sulfuric acid, hydrochloric acid, acetic acid, paracetic acid, citric acid, glycolic acid, formic acid, and mixtures or combinations thereof.

4. The composition of claim 2, wherein the surfactant or detergent is selected from the group consisting of: Brite Cleanse®, CHAPS, Pluronic® F-68, NP-40, sodium dodecyl sulfate (SDS), polysorbate 20, a saponin, Triton® X-100, sarkosyl, DetBuild®, and mixtures of combinations thereof.

5. The composition of claim 1, wherein the enzyme is isolated from an organism of the Archaea domain.

6. The composition of claim 5, wherein the enzyme is isolated from an organism of the Sulfolobales order.

7. The composition of claim 1, wherein the enzyme is selected from the group consisting of: a protease, a lipase, a cellulase, a hemicellulase, a glycoside hydrolase, an endoprotease, a carboxyesterase, an amylase, an alpha-amylase, an endoglucanase, an endopullulanase, a PNGase, a trehalase, a pullulanase, a peptidase, a signal peptidase, a xylanase, a cellobiohydrolase (CBH), a β-glucosidase, a peroxidase, a phospholipase, an esterase, a cutinase, a pectinase, a pectate lyase, a mannanase, a keratinase, a reductase, an oxidase, a phenoloxidase, a lipoxygenase, a ligninase, a tannase, a pentosanase, a malanase, a β-glucanase, an arabinosidase, a hyaluronidase, a chondroitinase, a laccase, a xyloglucanase, a xanthanase, an acyltransferase, a galactanase, a xanthan lyase, a xylanase, an arabinase, and combinations thereof.

8. The composition of claim 1, further comprising a food-safe additive.

9. The composition of claim 1, wherein the composition is effective for cleansing and/or sanitizing at a temperature of from about 50° C. to about 110° C.

10-14. (canceled)

15. The composition of claim 1, wherein the composition is effective for cleansing and/or sanitizing at a pH of from about 0.5 to about 7.

16-21. (canceled)

22. A method of cleaning a soiled surface, comprising:
(a) providing the composition of claim 1; and
(b) applying or contacting the composition with the surface,
wherein the temperature of the composition upon application to the surface ranges from about 50° C. to about 110° C. and has a pH of from about 0.5 to about 7.0, and
wherein the method results in at least about 25% of soil removal from the surface.

23-25. (canceled)

26. The method of claim 22, wherein the composition is applied to the surface for a duration of time ranging from about 5 minutes to about 180 minutes.

27-34. (canceled)

35. The method of claim 22, wherein the composition is applied to the surface for a duration of time of at least about 5 minutes.

36. The method of claim 22, further comprising a step (c) of recovering the composition.

37. (canceled)

38. (canceled)

39. The method of claim 36, further comprising a step (c) step (d) of applying or contacting the recovered composition with one or more additional surfaces to be cleaned.

40-51. (canceled)

52. A method of sanitizing a surface, comprising:
(c) providing the composition of claim 1; and
(d) applying or contacting the composition with the surface,
wherein the temperature of the composition upon application to the surface ranges from about 50° C. to about 110° C. and has a pH of from about 0.5 to about 7.0, and
wherein at least 95% of living organisms on the surface are eliminated and/or killed after applying the composition to the surface.

53-58. (canceled)

59. The method of claim 52, wherein the composition is applied to the surface for a duration of time ranging from about 5 minutes to about 180 minutes.

60-67. (canceled)

68. The method of claim 52, wherein the composition is applied to the surface for a duration of time of at least about 5 minutes.

69. The method of claim 52, further comprising a step (c) of recovering the composition.

70. (canceled)

71. The method of claim 69, further comprising a step (d) of applying or contacting the recovered composition with one or more additional surfaces to be sanitized and/or disinfected.

72-84. (canceled)

85. The method of claim 52, further comprising a step:
(b1) assaying the surface after contacting the composition with the surface to determine that the surface is free of biofilm residue.

86. A method of removing a biofilm residue from a surface, comprising:
(e) providing the composition of claim 1; and
(f) contacting the composition with the surface,
wherein the temperature of the composition upon application to the surface ranges from about 50° C. to about 110° C. and has a pH of from about 0.5 to about 7.0, and
wherein the biofilm residue is reduced and/or eliminated from the surface after applying the composition to the surface.
